SEQUENCE LISTING (15 sequences)

SEQ ID NO: 1 - DNA, 11 bp, synthetic construct
  source 1..11: mol_type = other DNA
agcactgcacc

SEQ ID NO: 2 - DNA, 11 bp, synthetic construct
  source 1..11: mol_type = other DNA
accagagatgc

SEQ ID NO: 3 - DNA, 11 bp, synthetic construct
  source 1..11: mol_type = other DNA
ggaatgaagga

SEQ ID NO: 4 - DNA, 11 bp, synthetic construct
  source 1..11: mol_type = other DNA
cagactccggt

SEQ ID NO: 5 - DNA, 11 bp, synthetic construct
  source 1..11: mol_type = other DNA
tgggtaagacg

SEQ ID NO: 6 - DNA, 11 bp, synthetic construct
  source 1..11: mol_type = other DNA
ccaaacactgc

SEQ ID NO: 7 - DNA, 11 bp, synthetic construct
  source 1..11: mol_type = other DNA
acgtgctgcta

SEQ ID NO: 8 - DNA, 11 bp, synthetic construct
  source 1..11: mol_type = other DNA
cgccaatattt

SEQ ID NO: 9 - DNA, 30 bp, synthetic construct
  source 1..30: mol_type = other DNA
cacggaatgagggccaggacagggggccgg

SEQ ID NO: 10 - DNA, 30 bp, synthetic construct
  source 1..30: mol_type = other DNA
tgaggacccgaggtcgcaggtggatctctt

SEQ ID NO: 11 - RNA, 22 nt, synthetic construct
  source 1..22: mol_type = other RNA
  modified_base 3: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 19: mod_base = OTHER; note = uracil
  modified_base 22: mod_base = OTHER; note = uracil
ggtgcagtgctgcatctctggt

SEQ ID NO: 12 - RNA, 21 nt, synthetic construct
  source 1..21: mol_type = other RNA
  modified_base 1: mod_base = OTHER; note = uracil
  modified_base 4..5: mod_base = OTHER; note = uracil
  modified_base 8..9: mod_base = OTHER; note = uracil
  modified_base 19: mod_base = OTHER; note = uracil
  modified_base 21: mod_base = OTHER; note = uracil
tccttcattccaccggagtct

SEQ ID NO: 13 - RNA, 22 nt, synthetic construct
  source 1..22: mol_type = other RNA
  modified_base 3: mod_base = OTHER; note = uracil
  modified_base 5..6: mod_base = OTHER; note = uracil
  modified_base 16: mod_base = OTHER; note = uracil
  modified_base 18..20: mod_base = OTHER; note = uracil
cgtcttacccagcagtgtttgg

SEQ ID NO: 14 - RNA, 22 nt, synthetic construct
  source 1..22: mol_type = other RNA
  modified_base 1: mod_base = OTHER; note = uracil
  modified_base 11: mod_base = OTHER; note = uracil
  modified_base 15: mod_base = OTHER; note = uracil
  modified_base 17..18: mod_base = OTHER; note = uracil
tagcagcacgtaaatattggcg

SEQ ID NO: 15 - RNA, 60 nt, synthetic construct
  source 1..60: mol_type = other RNA
  modified_base 10: mod_base = OTHER; note = uracil
  modified_base 12: mod_base = OTHER; note = uracil
  modified_base 15: mod_base = OTHER; note = uracil
  modified_base 21: mod_base = OTHER; note = uracil
  modified_base 24..25: mod_base = OTHER; note = uracil
  modified_base 29: mod_base = OTHER; note = uracil
  modified_base 37: mod_base = OTHER; note = uracil
  modified_base 43: mod_base = OTHER; note = uracil
  modified_base 50: mod_base = OTHER; note = uracil
  modified_base 55: mod_base = OTHER; note = uracil
  modified_base 58: mod_base = OTHER; note = uracil
ccggccccctgtcctggccctcattccgtgaagagatccacctgcgacctcgggtcctca

(19) United States
(12) Patent Application Publication
(10) Pub. No.: US 2024/0307867 A1
(43) Pub. Date: Sep. 19, 2024

(54) FIBER ARRAYS

(71) Applicant: Early is Good, Inc., Indianapolis, IN (US)

(72) Inventors: Thakshila Udayakanthi HABARAKADA LIYANAGE, Indianapolis, IN (US); Asel Ananda HABARAKADA LIYANAGE, Indianapolis, IN (US)

(21) Appl. No.: 18/174,463

(22) Filed: Feb. 24, 2023

(51) Int. Cl. B01L 3/00; B82Y 30/00; G01N 21/65; G01N 33/00; G01N 33/543

(52) U.S. Cl. CPC B01L 3/502707; B82Y 30/00; G01N 21/658; G01N 33/0031; G01N 33/54346; G01N 2201/0833

(57) ABSTRACT

The present invention includes composition and method that can be used in analyte detection. Certain embodiments are directed to fiber arrays comprising plurality of nanoparticles linked to one or more fibers, where a fiber is linked to the nanoparticle through a linker. The fiber array can be provided in different sizes and shapes.

### FIELD OF THE INVENTION

The present invention relates to fiber arrays and spectrometric detection.

### REFERENCE TO AN ELECTRONIC SEQUENCE LISTING

The contents of the electronic sequence listing (068469-P3US1.xml; Size: 29,600 bytes; and Date of Creation: Feb. 24, 2023) is herein incorporated by reference in its entirety.

### BACKGROUND OF THE INVENTION

Analyte detection and quantification is useful in many areas including detecting food contaminants, detecting pathogens, detecting environmental toxins, biological analysis, and detecting biomarkers associated with a particular disease or order. Biomarker detection and quantification can be used to identify subjects who would benefit from therapeutic intervention and to guide treatment. For example, analyte detection can be used in cancer detection and treatment. Clinical diagnosis is very important in early detection, monitoring tumor progression, and therapeutic response. Bellassia et al., 2019 Front. Chem. 7:570 mention the use localized surface plasmon resonance and single-stranded DNA, for detecting low levels of miR-182, miR-10b, miR-143 and miR-145 associated with bladder cancer. U.S. Patent publication No. 2021/0140911 includes biosensors and systems that can be used for analyte detection.

Li et al., Sensors (2018), 18, 3295 mentions integration of functional nanomaterial with optical micro/nanofibers.

U.S. Patent publication No. 2012/005869 mentions fibrous substrates functionalized with particles.

Pilot et al., Biosensors (2019), 9, 57; doi: 10.3390/bios9020057S, reviews surface-enhanced Raman techniques and includes examples of structures for carrying out surface-enhanced Raman.

Luan et al. Light and Science (2018) 7:29, mentions conformal elastomeric film with absorbed nanoparticles.

### BRIEF SUMMARY OF THE INVENTION

The present invention features a fiber array comprising a plurality of nanoparticles attached to one or more fibers; and nanoparticle combinations. The different members of the plurality of nanoparticles comprise a nanoparticle attached to a fiber through a linker. The fiber array can be provided in different sizes and shapes.

Thus, a first aspect of the present invention relates to a fiber array comprising a plurality of a nanoparticles attached to one or more fibers, wherein each nanoparticle of the plurality of the nanoparticles is attached to a fiber of the one or more fibers at a different location through a linker wherein at least one of either:
a) the linker is attached to the fiber through a silane coupling agent.
b) the nanoparticle further comprises an analyte capture molecule.
c) the linker is covalently attached to the nanoparticle;
d) the linker is 20 Da to 500 KDa; and/or
e) the fiber array comprises at least two fibers.

Reference to a “fiber array”, indicates one or more fibers making up the array provides for two or more segments and/or two or more fibers providing a width and length. A single fiber can provide for an array by looping back and/or around.

A three-dimensional fiber array provides an additional height dimension, where one or more fibers or fiber segment is above another fiber or fiber segment.

Each of the nanoparticles making of the plurality of the nanoparticles is attached to fiber though its own linker. Thus, the plurality of the nanoparticles comprises separate nanoparticle-linkers attached to the one or more fibers at different locations. In addition to the indicated plurality of the nanoparticles, reference to comprises allow for additional nanoparticles that may be attached to the one or more fibers where, for example, the additional nanoparticles have a different composition, are not attached by a linker and/or are attached by a different linker than that present in the plurality of nanoparticles.

Reference to plurality of the nanoparticles indicates the presence of at least about 100 nanoparticles, where each nanoparticle is attached to a fiber at a different location through its own linker. The number, density, and coverage of the nanoparticles on the fiber array can vary depending upon, for example, the desired dimensions and application. In different embodiments the plurality of the nanoparticles provides at least 500, at least about 1,000, at least 5,000 or at least 10,000 nanoparticles, where each nanoparticle is attached to a fiber, or multiple fibers, at a different location through its own linker.

Another aspect of the present invention relates to methods involving a fiber array provided herein to detect the presence of one or more analyte and or signal amplification. Detecting the presence of one on more analytes can be qualitative or quantitative.

Different methods can be performed taking in to account the composition of a particular array and the use of additional components. Example of different types of methods include use of localized surface plasmon resonance (LSPR), surface enhanced Raman spectroscopic (SERS), enzyme-linked immunoassay applications (ELISA), Fourier transform infrared spectroscopic (FTIR), bioluminescence, fluorescence, chemiluminescence and paper-based mass spectrometry.

Additional aspects include a nanoparticle combination, and uses of different members of the combination, where the combination comprises:
a) a first plasmonic nanoparticle comprising a first analyte binding molecule that specially binds to analyte, wherein the first analyte binding molecule further comprises a first energy transfer molecule, and
b) a second plasmonic nanoparticle comprising a second analyte binding molecule that specially binds to the analyte, wherein the second analyte binding molecule further comprises a second energy transfer molecule;
   wherein either the first energy transfer molecule is a donor and the second energy transfer molecule is an acceptor or the first energy transfer molecule is an acceptor and second energy transfer molecule is a donor.

The nanoparticle combination can be provided together, for example, in a kit; or be provided and used separately. In certain embodiments, the first plasmonic nanoparticle is attached to a fiber array through a linker. In certain embodiments, the first plasmonic nanoparticle is attached to a fiber array without a linker.

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

FIG. 1 provides a scanning electron microscope image of two three-dimensional fiber arrays.

FIG. 2 provides a scanning electron microscope of fiber illustrating a high density of nanoparticles.

FIG. 3 illustrates a nanoparticle functionalized with different functional groups.

FIG. 4 illustrates examples of nanoparticle functional groups and nanoparticles joined to a siliconized fiber through different functional groups.

FIGS. 5A, 5B and 5C illustrate attachment of nanoparticle to a fiber using different types of chemistry. FIG. 5A illustrates thiol and amine chemistry. FIG. 5B illustrates amide and click chemistry. FIG. 5C illustrates ester chemistry.

FIG. 6A and FIG. 6B illustrates energy detection using energy transfer molecules present on different analyte binding molecules

FIGS. 7A and 7B show calibration plots measuring luminescence and fluorescence for different analytes. FIG. 7A illustrates CRH, ANAX10, ABL1, IGF2 and UPKB1 luminescence. FIG. 7B illustrates CRH, ANAX10, ABL1, IGF2 and UPKB1 fluorescence.

FIGS. 8A and 8B show calibration plots measuring luminescence and fluorescence for different analytes. FIG. 8A illustrates miR-200c (200c), miR-16-1 (16-1), miR-205 (205), miR-143 (143) and long non-coding RNA (UCA) luminescence. FIG. 8B illustrates miR-200c (200c), miR-16-1 (16-1), miR-205 (205), miR-143 (143) and long non-coding RNA (UCA) fluorescence.

FIGS. 9A and 9B show calibration plots measuring luminescence and fluorescence for different analytes. FIG. 9A illustrates NMP22, HCFHrp (hcfHRP) and P53 luminescence. FIG. 8B illustrates NMP22, HCFHrp (hcfHRP) and P53 fluorescence.

### DETAILED DESCRIPTION OF THE INVENTION

The present features a fiber array comprising a plurality of nanoparticles attached to one or more fibers; and nanoparticle combinations. The different members of the plurality of nanoparticles comprise a nanoparticle attached to a linker, where different nanoparticle-linkers are attached to a fiber at different location. The fiber array can be provided, for example, in different sizes, shapes, and composition, and has a variety of different uses.

Different sizes, shapes and compositions can be selected, for example, to facilitate different assays, detection techniques, assay formats and equipment. Examples of different sizes and shapes include fiber arrays fitting in standard 6, 12, 96, or 384 wells (e.g., 5, 6, 10, 13, 14 or 20 diameter wells), a petri dish, glass slide (e.g., having a width of about 25 mm to about 90 mm) or microfluidic plate.

In certain embodiments, the fiber array is adopted for multi-readouts; are used with black or white well plates (e.g., fluorescence, chemiluminesence, or bioluminscense) readouts; are used with transparent well-plates (e.g., local resonance surface application); and/or comprise an adhesive to fix the array on to a surface.

Uses of the fiber array include spectrometric methods detecting the presence of an analyte. The particular type of spectrometric method employed takes into account the nanoparticle structure and other components that may be present.

The fiber array can be provided as ready to use in a particular assay or in a form that can be customized for a particular assay. A ready to use to fiber array provides a form that can be used without further processing. Examples of a ready to use fiber array is an array where nanoparticles of the plurality nanoparticles further comprise an analyte capture molecule; or the array is used for signal amplification in methods where the analyte is captured to the array. An example of a form that can be customized for a particular assay, is an array comprising functional groups to which analyte detection molecules can be attached.

Subject areas involving analyte detection include food safety, for example, detecting contaminants such as pathogens, mycotoxins, plant and bio-marine toxins, toxic chemical, preservatives, and anti-oxidants; environment contaminants, such as pesticides, herbicides, aromatic compound, chemical mixtures, and toxic metal contaminants; human security, for example, explosive and chemical warfare detection; and health areas, for example detecting biomarkers associated with a particular disease or disorder, including certain polypeptides, proteins, DNA, RNA, exosomes and detecting infectious organisms. (See, for example, Camarca et al., Sensors (Basel). 2021 Jan. 29; 21(3):906.)

Therapeutic applications of detecting and quantifying biomarkers associated with a particular disease or order can include diagnostic, prognostic, and treatment guiding. Diagnostic applications include early detection, and obtaining information on a particular type of a disease or disorder and the severity or stage of a disease or disorder. Prognostic applications include prediction of disease outcome and treatment response. Treatment guiding relates to the type of treatment selected, monitoring treatment, and adjusting treatment based on particular analyte levels.

In certain embodiments, the analyte is a protein or polypeptide. Examples of protein and polypeptide analytes that can be detected include enzymes, substrates, antibodies, protein hormones, receptors, and cytokines.

In certain embodiments, the analyte is a nucleic acid. Examples of nucleic acid that can be detected include RNA and DNA, such as mRNA, tRNA, siRNA, lncRNA, piwiRNA, snoRNA, tircRNA, mitochondrial RNA, non-coding RNA, miRNA, cDNA, genomic DNA, and enzymes (e.g., ribozymes).

In certain embodiments, the analyte is extracellular RNA, such as extracellular mRNA.

In certain embodiments, the analyte is an extracellular vesicle, such as extracellular exosomes.

In certain embodiment, the analyte is a virus. Examples of viruses that can be detected include, pathogenic viruses infecting a subject, plant, agricultural product, or food; or present in the environment (for example water supply and ventilation).

In certain embodiments, the analyte is a cell. Examples of cells that can be detected include bacterial combinations (e.g., present in the gut); pathogenic bacteria infecting a subject, plant, agricultural product, or food; or present in the environment (for example water supply and ventilation).

In certain embodiments, the analyte is a polysaccharide. Examples of polysaccharides that can be detected include polysaccharides associated with bacteria or a particular disease or disorder in a subject.

In certain embodiments, the analyte is a lipoprotein. Examples of lipoprotein that can be detected include high density lipoprotein, low density, and bacterial lipoproteins.

In certain embodiments, the analyte is a lipid. Examples of lipids that can be detected include cholesterol and hormones.

In certain embodiments, the analyte is a therapeutic compound. Examples of therapeutic compounds include small molecules, and large molecules such as antibodies and proteins.

In certain embodiments, the analyte is an environmental pollutant. Examples of pollutants include air pollutants such as nitrogen oxides, and sulfur dioxide; water pollutants such as pesticides, heavy metals, and per- and polyfluoroalkyl substances (PFAS); and soil pollutants such as pesticides, heavy metals, and PFAS.

In certain embodiments, the analyte is an illegal drug.

Reference to “subject” indicates a mammal, including a human; non-human primates such as apes, gibbons, gorillas, chimpanzees, orangutans, and macaques; domestic animals such as dogs and cats; farm animals such as poultry, ducks, horses, cows, goats, sheep and pigs; and experimental animals such as mice, rats, rabbits, and guinea pigs. A preferred subject is a human.

The terms “nucleic acid” and “polynucleotide” are used interchangeably herein to refer to a nucleotide polymer without regard to function or size. Nucleic acid and polynucleotides contain at least two nucleotides and include deoxyribonucleic acid (DNA) and ribonucleic acid (RNA); and in some cases, DNA or RNA derivatives having nitrogenous bases able to hydrogen bond to DNA or RNA, where a derivative contains a modified nucleobase, sugar moiety and/or phosphodiester linkage. In discussing nucleic acids and polynucleotides, unless otherwise indicated, a provided sequence is in the 5′ to 3′ direction.

The terms “polypeptide,” “protein” and “peptide” can be used interchangeably to refer to an amino acid sequence without regard to function. Polypeptides and peptides contain at least two amino acids, while proteins contain at least about 10 amino acids. The provided amino acids include naturally occurring amino acids and amino acids provided by cellular modification.

The singular forms “a,” “an,” and “the” include plural reference unless the context clearly dictates otherwise.

The conjunctive term “and/or” between multiple recited elements encompasses both individual and combined options. For instance, where two elements are conjoined by “and/or”, a first option refers to the applicability of the first option without the second, a second option refers to the applicability of the second option without the first, and a third option refers to the applicability of the first and second options together. Any one of the options is understood to fall within the meaning and therefore satisfy the requirement of the term “and/or”. Concurrent applicability of more than one of the options is also understood to fall within the meaning of the term “and/or.”

Unless clearly indicated otherwise by the context employed the terms “or” and “and” have the same meaning as “and/or”.

Reference to terms such as “including”, “for example”, “e.g.,”, “such as” followed by different members or examples, are open-ended descriptions where the listed members or examples are illustrative and other members or examples can be provided or used.

Reference to “comprise”, and variations such as “comprises” and “comprising”, used with respect to an element or group of elements is open-ended and does not exclude additional unrecited elements or method steps. Terms such as “including”, “containing” and “characterized by” are synonymous with comprising. In the different aspects and embodiments described herein reference to an open-ended term such as “comprising” can be replaced by “consisting” or “consisting essentially of”.

Reference to “consisting of” excludes any element, step, or ingredient not specified in the listed claim elements, where such element, step or ingredient is related to the claimed invention.

Reference to “consisting essentially of” limits the scope of a claim to the specified materials or steps and those that do not materially affect the basic and novel characteristic(s) of the claimed invention.

The term “about” refers to a value within 10% of the underlying parameter (i.e., plus or minus 10%). For example, “about 1:10” includes 1.1:10.1 or 0.9:9.9, and “about 5 hours” includes 4.5 hours or 5.5 hours. The term “about” at the beginning of a string of values modifies each of the values. In an embodiment, the term about refers to a range within 5% of the underlying parameter.

All numerical values or numerical ranges include integers within such ranges and fractions of the values or the integers within ranges unless the context clearly indicates otherwise. Reference to an integer with more (greater) or less than includes numbers greater or less than the reference number, respectively.

Various references including articles and patent publications are cited or described in the background and throughout the specification. Each of these references is incorporated by reference in their entirety. None of the references are admitted to be prior art with respect to any inventions disclosed or claimed. In some cases, particular references are indicated to be incorporated by reference herein to highlight the incorporation.

The definitions provided herein, including those in the present section and other sections of the application, apply throughout the present application.

Unless defined otherwise, all technical and scientific terms used herein have the same meaning commonly understood to one of ordinary skill in the art to which this invention pertains.

The description has been separated into various sections and paragraphs, and provides examples of various embodiments. These separations should not be considered as disconnecting the substance of a paragraph or section or embodiments from the substance of another paragraph or section or embodiment. The provided descriptions have broad application and encompasses all the combinations of the various sections, paragraphs and sentences that can be contemplated. The discussion of any embodiment is meant only to be exemplary and is not intended to suggest the scope of the disclosure, including the claims (unless otherwise provided in the clams), is limited to these examples.

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, embodiments that are not expressly excluded in the instant invention are nevertheless disclosed herein.

### I. Fiber Arrays

Fiber arrays can comprise using different types of nanoparticles, different types of fibers, and different types of linkers. The fiber array can be produced with varying two dimensions of width and length; and in three-dimensions with different heights/thickness. Considerations for fiber height include type of fiber, nanoparticle and the potential impact of height on signal production and, if applied, excitation throughout the fiber array. For localized surface plasmon resonance it is more important for light to passthrough the array and an optically transparent fiber is preferred. For other applications such as those involving fluorescence, luminescence, chemiluminescence, and surface enhanced Raman, less or non-transparent substrates can be used.

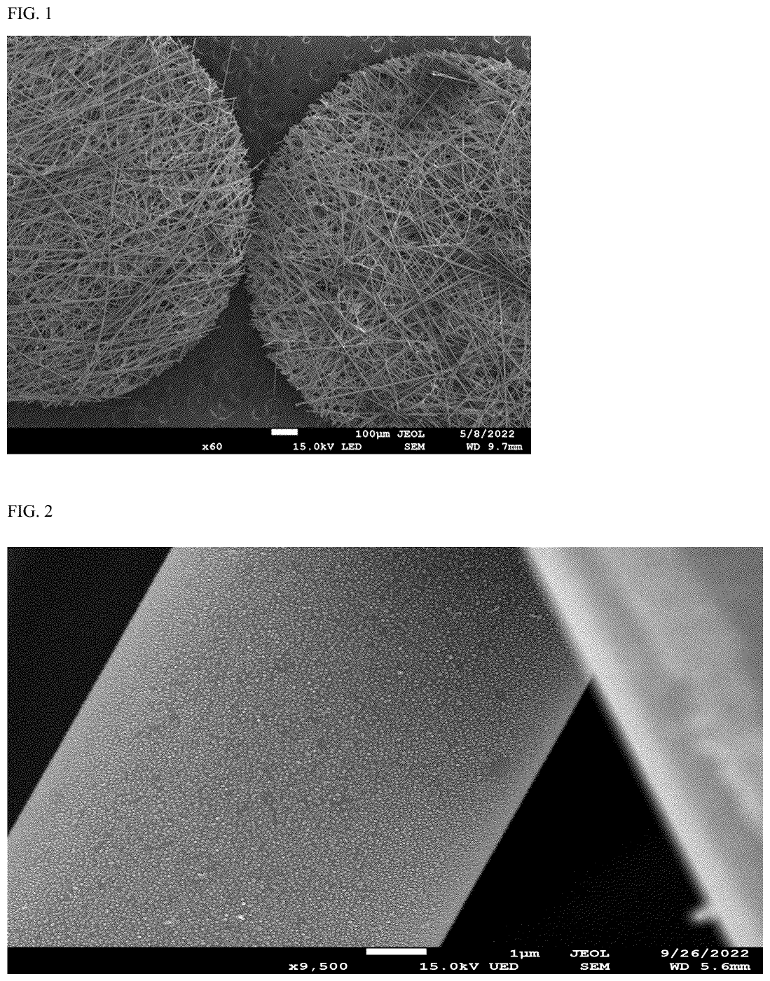

In certain embodiments the fiber array is a three-dimensional array having a height of 0.05 mm to 100 mm; 0.1 mm to 100 mm; 0.1 mm to 10 mm; or 0.2 mm to 1 mm. FIG. 1 illustrates a three-dimensional fiber array with a height of about 0.43 mm.

In certain embodiments the one or more fibers comprises at least 2, at least 5, at least 10, at least 100, or at least 500 fibers.

The density of nanoparticles on a particular fiber can vary. In certain embodiments, the the plurality of nanoparticles provides for about 4% to about 95% coverage over a nanofiber area of at least 14 μm². Reference to a region of at least 14 μm² indicates the presence of one or more regions having an area of at least 14 μm².

In further embodiments, the plurality of the nanoparticles provides for about 4% to about 95% coverage over a region of at least 24 μm², at least 50 μm², at least 100 μm² or at least 5000 μm² of the one or more fibers; the plurality of the nanoparticles provides for about 25% to about 95% coverage over a region of at least 12 μm², at least 24 μm², at least 50 μm², at least 100 μm² or at least 5000 μm² of the one or more fibers; the plurality of the nanoparticles provides for about 50% to about 95% coverage over a region of at least 12 μm², at least 24 μm², at least 50 μm², at least 100 μm² or at least 5000 μm² of the one or more fibers; the plurality of the nanoparticles provides for about 75% to about 95% coverage over a region of at least 12 μm², at least 24 μm², at least 50 μm², at least 100 μm² or at least 5000 μm² of the one or more fibers; the plurality of the nanoparticles provides for about 75% to about 95% coverage over a region of at least 12 μm², at least 24 μm², at least 50 μm², at least 100 μm² or at least 5000 μm² of the one or more fibers; the plurality of the nanoparticles provides for about 50% to about 75% coverage over a region of at least 12 μm², at least 24 μm², at least 50 μm², at least 100 μm² or at least 5000 μm² of the one or more fiber.

In certain embodiments wherein the plurality of the nanoparticles provides a region of about 10 to about 100 nanoparticles over a region of at least 25 μm² of the one or more fibers. In further embodiments the plurality of the nanoparticles provides a region of about 25 to about 75 nanoparticles over a region of at least 25 μm².

FIG. 2 is a scanning electron micrograph of a fiber providing for high density of nanoparticles. The figure shows Au spherical nanoparticles having a diameter of about 660 nm and includes a region of 0.25 μm² containing 66 nanoparticles. The figure also shows 75% coverage.

Calculation of density and percent coverage can be carried out using a scanning electron micrograph. The area of coverage can be carried out as follow: (1) count the number of nanoparticles in a selected fiber area (e.g., using Image J software); (2) determine the nanoparticle area based on the nanoparticle diameter; (3) multiply the total number of nanoparticles by the individual area to obtain the total area of the nanoparticles; and (4) to find the total coverage, divide the total area of the nanoparticles by the area of the substrate and express the result as a percentage: Total Coverage=(Total Area of Nanoparticles/Area of Substrate)*100%.

In certain embodiments, the coverage is about 4% to about 95%, about 25% to about 95%, about 50% to about 95% coverage, about 75% to about 95%, about 75% to about 95%, or about 50% to about 75% over the total fiber array. Percent coverage over the total fiber array can be provided, for example, by determining coverage at representative locations (e.g., at least 10, 25 or 50 different locations of at least about at least 24 μm² or at least 50 μm².

In certain embodiments, the fiber array fits within a well having a diameter of 5, 6, 10, 13, 14 or 20 mm and a depth of 11 mm.

Nanoparticles can be provided in different shapes having a variety of different length, width and size. Examples of different shapes include spheres, stars, prisms, cubes, triangles, spikes and rods. In certain embodiments nanoparticles have a size less than about 5000 nm across its longest dimension. In different embodiments, the nanoparticle is about 1 nm to 5000 nm, about 1 nm to about 500 nm, about 50 nm to about 200 nm, about 1 nm to about 100 nm, about 10 nm to about 100 nm, about 2.5 nm to about 100 nm across its longest dimension.

In certain embodiments, the nanoparticle is spherical, where in different embodiments, the nanoparticle is about 1 nm to 5000 nm, about 1 nm to about 500 nm, about 50 nm to about 200 nm, about 1 nm to about 100 nm, about 10 nm to about 100 nm, or about 2.5 nm to about 100 nm in diameter.

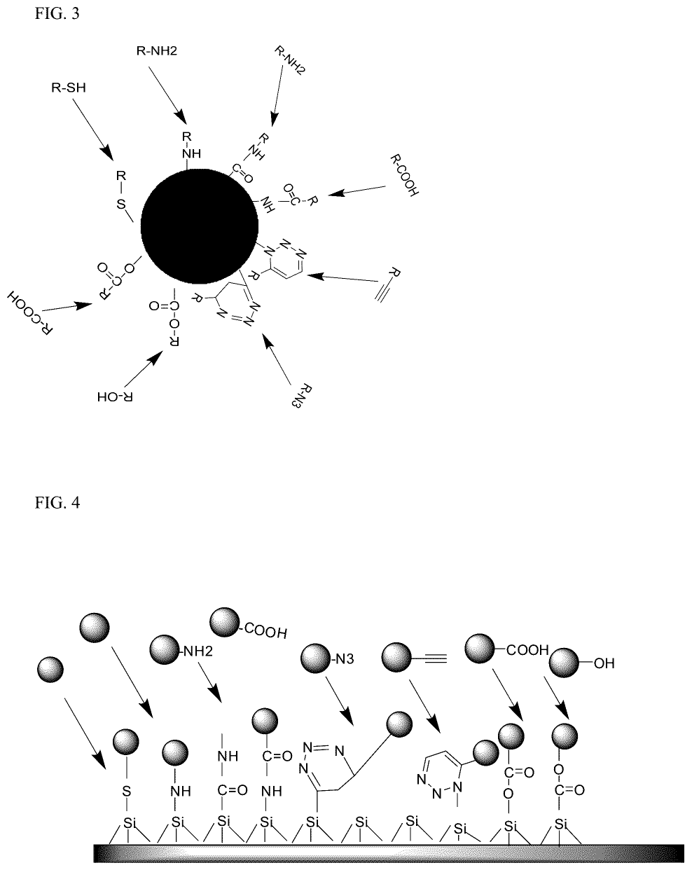

Nanoparticles can be functionalized with different groups to facilitate linker attachment. FIG. 3 illustrates a nanoparticle functionalized with different functional groups, where R indicates a group subject to additional functionalization and the location where additional functionalization can take place. In certain embodiments, the nanoparticle is functionalized with a molecule having an R functional group selected from hydroxyl, alkyne, sulfhydryl, amine, amide, carboxylic, and azido-triazine. The R functional group can be further capped with a ligand and further derivatized. In certain embodiments, the R functional group is capped with ligand provided in Table 1. Table 1 compounds referencing “n”, provide for n=1 to n=10,000.

```
TABLE 1

  PEG(n)
  mPEG(n)-PLGA-SH
  CTAB

  DSPE-PEG(n)-Ald
  DSPE
  DSPE-PEG(n)-azide
  DOPE-PEG(n)-COOH
  DSPE-PEG(n)-NHS
  DSPE-PEG(n)-SH
  1,2-Distearoyl-sn-glycero-3-phosphoethanolamine
  Tri(propargyl-PEG(n)-NHCO-ethyloxyethyl)amine
  N-(Boc-PEG3)-N-bis-(PEG(n)-Amino-Tri-(Propargyl-PEG(n)-ethoxymethyl)-methane)
  Tetra(3-methoxy-N-(PEG(n)-prop-2-ynyl)propanamide) Methane
  endo-BCN-PEG(n)-acid
  DBCO-PEG(n)-acid
  DBCO-NHCO-PEG(n)-acid
  Gly-Gly-Gly-PEG(n)-DBCO
  Sulfo DBCO-PEG(n)-acid
  Propargyl-PEG(n)-acid
  DBCO-PEG(n)-amine TFA salt
  Propargyl-PEG(n)-CH2CO2H
  Propargyl-PEG(n)-amine
  t-Boc-N-Amido-PEG(n)-propargyl
  Propargyl-PeG(n)-alcohol
  Aminooxy-PEG1-propargyl HCl salt
  N-(Amino-PEGn)-N-bis(PEG3-acid)
  Amino-PEG(n)-amine
  N-(Amino-PEG(n))-N-bis(PEG3-azide)
  m-PEG2-amine
  Amino-Tri-(Azide-PEG(n)-ethoxymethyl)-methane
  N-(Amine-PEG(n))-N-bis-(PEG(n)-Amino-Tri-(Propargyl-PEG(n)-ethoxymethyl)-methane)
```

In certain embodiments, binding to the nanoparticle is through a thiol, phosphene, amine, a polymer, or silica. (See, e.g., Mahota et al., (2019) 3 Biotech 9:57, illustrating functionalization using gold nanoparticles).

In certain embodiments the nanoparticle is functionalized with a silane modifier. Silane modifiers are described by, for example, Ahangaran and Navarchian (2020) Advances in Colloid and Interface Science 286:102209, and section I.E. infra.

In certain embodiments, the nanoparticle further comprises an analyte capture molecule and/or signal generating molecule. Reference to “signal generating molecule” indicates a molecule able to generate a detectable signal or participate in signal generation. Examples of signal generating molecules include fluorescent molecules, plasmonic material, metals, enzymes, phosphorescence, latex bead, and chelates.

In certain embodiments the fiber array provides for and/or enhances spectrometric emissions. Spectrometric emissions can broadly be defined as changes in electromatic radiation as a result of interaction with a substance.

### I.A. Plasmonic Nanoparticles

Plasmonic nanoparticles comprises a material able to generate surface plasmon resonance. Surface plasmon resonance is generated by an electromagnetic surface wave, due to a collective oscillation of free electrons propagating parallel to an interface region. The binding of analytes to such material generates surface plasmon resonance leading to a change in refractive index, which can be identified as a wavelength shift or surface plasmon resonance intensity.

Plasmonic nanostructures can also enhance spectrometric emission from other substances, such as fluorescent, bioluminescent, and chemiluminescent emission, and surface-enhanced Raman scattering. Detection of fluorescent emission can be carried out using appropriate fluorescent energy transfer pairs and an excitation source. In the case of chemiluminescence, initial generation of electromagnetic radiation is chemically generated, and resonance can be measured using an appropriate fluorophore. Bioluminescence is a type of chemiluminescence where the initial electromagnetic radiation is enzymatically generated, and resonance is measured using an appropriate fluorophore. (See, for example, Fereja et al., ISRN Spectroscopy (2013) Volume 2013, Article ID 230858.)

Surfaced-enhanced Raman scattering can be used to identify vibrational fingerprints of particular molecules. In surfaced-enhanced Raman scattering, amplification of electromagnetic fields generated by the excitation of localized surface plasmons enhance the Raman scattering. Surfaced-enhanced Raman techniques including use of plasmonic structures and assay formats are provided in Pilot et al., Biosensors (2019), 9, 57; doi: 10.3390/bios9020057 hereby incorporated by reference in its entirety.

Surface plasmon can be confined and excited on sub-wavelength size nanoparticles with a specific frequency known as localized surface plasmon resonance. The localized surface plasmon plasmonic nanostructure is affected by the morphology, size, composition, and distance between adjacent nanostructures. Examples of different three-dimensional shapes include spheres, stars, prisms, cubes, triangles, spikes and rods.

Localized surface plasmon resonance nanoparticles can be based on different platforms and made of different materials. The array, for example, can comprise metals such as rhenium, ruthenium, rhodium, palladium, silver, osmium, iridium, platinum, gold and alumni; and combinations of materials. (See, for example, Park et al. Biosensors (Basel), 2022, 17; 12(3):180. doi: 10.3390/bios12030180; and Osborne and Pikramenou (2015) Faraday Diss., 185:291.) Examples of combinations include nickel, gold-silver alloy nanoparticles (Au—Ag), gold-palladium alloy nanoparticles (Au—Pd), gold-copper alloy nanoparticles (Au—Cu), silver-copper alloy nanoparticles (Ag—Cu), gold-silver-copper alloy nanoparticles (Au—Ag—Cu), gold-silver-palladium alloy nanoparticles (Au—Ag—Pd), metal oxide nanoparticles (e.g., titanium dioxide and zinc oxide), and metal-semiconductor nanoparticles (e.g., gold-silicon and silver-silicon).

Additional references describing surface plasmon resonance, detection techniques, materials, and configurations include Park et al. Biosensors (Basel), 2022, 17; 12(3):180, doi: 10.3390/bios12030180; Camarca et al., Sensor 2021, 21, 906, hypertext//doi.org//10.3390/s2103906; Kim et al., Sensors 2021, 21, 3191, hypertext//doi.org//10.3390/s21093191; and Liu and Zhang, Micromachines 2021, 12, 826, hypertext//doi.org/10.3990/mil2070826; each of which are hereby incorporated by reference in its entirety herein.

### I.B Semiconductor

Semiconductor nanoparticles provide a quantum confinement effect, which leads to spatial enclosure of electronic charge carriers. Semiconductor size and shape can be varied to obtain energy of discrete electronic energy states and optical transition. In certain embodiments, the nanoparticle is a quantum dot. Semiconductors, such as quantum dots, can be used, for example, in resonance energy transfer reactions such as a fluorescent, bioluminescent, and chemiluminescent. Examples of semiconductor material include ZnO, Co₃O₄, CeO₂, CoFe₂O₄, Fe₂O₃, Fe₃O₄, ZnO, TiO₂/IrOx, IrOx, TiO₂, Al₂O₃, CdSe, CdS, and MoS₂. (See, for example, Smith et al., Acc Chem Res. (2010) 43(2): 190-200; and Pisanic et al., (2014), 139(1):2968-2981; both of which are hereby incorporated by reference herein in their entirety.)

### I.C. Additional Nanoparticles

Examples of additional nanoparticles that can be attached to fiber include liposome, micelle, peptide, dendrimer, polymeric nanoparticles, and polymeric vesicles. In certain embodiments, such nanoparticles are further derivatized or loaded with signal generating molecules. (See, for example, Sforzi et al., (2020) Biology 9, 202, and Idrissi et al., (2018) J. Nanobiotechnol 16, 63, each of which is hereby incorporated by reference herein in its entirety.)

In certain embodiments the nanoparticle is a liposome. Liposomes are spherical structures composed of a phospholipid bilayer and an inner aqueous compartment. Different types of liposome configurations can be produced, and different types of phospholipids can be employed. Different phospholipids can vary, for example, in the phospholipid head group and fatty acid group. Examples of different groups include head groups such as phosphatidylcholine, phosphatidylinositol, phosphatidylserine, phosphatidylethanolamine, phosphatidic acid, cardiolipin, and phosphatidylglycerol; fatty acids of different sizes and degree of saturation; and type of bonding between glycerol and aliphatic chain. (Andra et al., BioNanoScience (2022) 12:274-291, and Sforzi et al., (2020) Biology 9, 202, each of which is hereby incorporated by reference herein in its entirety.)

In certain embodiments the nanoparticle is a micelle. A micelle is made up of hydrophobic and hydrophilic components. (Mathew et al., Int. J. Pharm Sci. Rev. Res. (2020) 61(2):36-39 and Hanafy et al., Cancers (Basel) (2018) 10(7):238, both of which are hereby incorporated by reference herein in their entirety.)

In certain embodiments the nanoparticle is peptide based. Different types of peptide nanoparticles can be produced using, for example, peptides able to self-assembly and form nanostructure. Examples of peptide nanoparticles include those containing linear short helical peptides, constrained peptides with crosslinkers, and peptide chains containing two helical coiled-coil segments connected by a linker. (See, for example, Li et al., (2022) Bioactive Materials 11:268-282; Katyal et al., (2019) ACS Biomater. Sci. Eng. 5:4132-4147; and Doll et al., (2015) J. Nanobiotechnol. 13, 73, each of which are hereby incorporated by reference herein in their entirety.)

In certain embodiments the nanoparticle is a dendrimer. A dendrimer is a nano-size radially symmetric molecule made of branched polymers. A dendrimer can be functionalized with different compounds and can be produced having different properties and structures. (Abbasi et al., Nanoscale Research Letters (2014) 9:247; and Bosnjakovic et al., (2012); both of which are hereby incorporated by reference herein.)

In certain embodiments the nanoparticle is a polymeric vesicle. Polymeric vesicles are similar to liposomes, but they are composed of synthetic or natural polymers instead of phospholipids. (Idrissi et al., (2018) J. Nanobiotechnol 16, 63, hereby incorporated by reference herein it its entirety.)

In certain embodiments the nanoparticle is a polymeric nanoparticle. Polymeric nanoparticles are composed of synthetic or natural polymers such as poly(lactic-co-glycolic acid) (PLGA), polyethyleneimine (PEI), and poly(¿-caprolactone) (PPCL). Additional components and method of production are illustrated by, for example, Zielińska et al., (2020) Molecules. August 15; 25(16):3731 (hereby incorporated by reference here in its entirety). Polymeric nanoparticles such as those described by Zielińska et al., for therapeutic applications, provide guidance on the nanoparticle itself where, for example, instead of loading with a therapeutic agent, the nanoparticle is loaded with a signal generating molecule.

### I.D. Fibers

Fiber arrays are made up of one or more fibers. An array can contain one type of fiber or different types of fibers. Preferred fibers will depend upon the particular application and preferably have one or more of the following properties: good uniformity, strength and modulus, low density, chemical resistance, little flexibility, biocompatibility, open porosity, wide compatibility, and hydrophilicity. For localized surface plasmon resonance and related applications, materials should be transparent. For other applications such as fluorescence and bioluminescent an opaque or less transparent fiber should be used.

In certain embodiments a fiber comprises glass, quartz, indium tin oxide, flexible plastic, polyester, nylon, polypropylene, polyacrylonitrile (PAN), polyimide (PI), polybenzimidazole (PBI), Aramid or a combination thereof.

In certain embodiments a fiber making up the fiber array has a diameter of about 1 nm to about 50 μm. In further embodiments the diameter is about 1.0 nm to about 500 nm; about 1.0 nm to about 100 nm, about 200 nm, about 300 nm or about 400 nm; about 0.1 nm to about 2000 nm; about 1.0 nm to about 100 nm; about 1 μm to about 50 μm; about 100 μm to about 200 μm; about 2 μm to about 12 μm; or about 7 μm.

In certain embodiments, the fiber comprises cross-linked silicone oxide or is a polymer. Cross-linked silicone oxide is the predominate component of glass fiber. In certain embodiments, the fiber is a glass fiber. Different types of glass fibers can be used to provide for the fiber array. Glass fibers, in addition to containing SiO₂, may contain other components such as B₂O₃, Al₂O₃, CaO, MgO, ZnO, TiO₂, Zr₂O₃, Na₂O, K₂O, Li₂O, Fe₂O₃, and/or F₂. (Wallenberger et al., (2001), ASM Handbook, Vol. 21: Composites (#6781G), hereby incorporated by reference herein in its entirety).

In certain embodiments the fiber is a polymer comprising polyethersulfone, polydimethylsiloxane (PDMS), nylon, polypropylene, polylactic acid, cellulose, polycarbonate, polyacrylamide, polyacylonitrile (PAN), polyvinyl alcohol, cellulose acetate, polyvinyl chloride, polyamine/polyurethane, polyvinylidene fluoride (PVDF), polyethylene terephthalate (PET), polymethyl methacrylate (PMMA), polystyrene (PS), epoxy resin, thermoplastic polyurethane, poly(lactic-co-glycolic acid) (PLGA), polyether amine PEA, or polyvinylpyrrolidone (PVP).

In certain embodiments, the fiber is optically transparent. Reference to optically transparent indicates an optical density of less than 3.0 with respect to light. Optical density (OD) can be calculated by the formula OD=log 10 (I₀/I), where I₀ is the incident optical intensity (optical intensity hitting the material) and I is the transmitted optical intensity (optical intensity transmitted by the material). In different embodiments, the OD is less than 2.5, 2.0, 1.5, 1.0 or 0.5.

Fibers can be produced using different techniques including electrospinning, electrospraying, and non-electron spinning approaches. In certain embodiments the polymer is produced by electrospinning. Electrospinning is based on the application of high voltage to produce charged jet; a polymer solution is pumped through a thin nozzle and the high electric field difference applied between the nozzle causes the “extraction” of the polymer solution that dries out before reaching the counter electrode forming the fiber. In electrospraying, particles are made from capillary breakup of jet/filament due to surface tension and low solution/suspension viscosity. (See, for example, Nadaf et al., RSC Adv., (2022) 12, 23808; Islam et al., SN Applied Sciences (2019) 1:1248; Li et al., Polymers (2021) 13, 676; and Pilot et al., Biosensors (2019), 9, 57; doi: 10.3390/bios9020057; each of which are hereby incorporated by referenced herein in their entirety.)

### I.E. Linker

A nanoparticle can be attached to fiber using different types of chemistry and different types of linkers. The linker provides a stable structure and can be made up of different groups joined together by different types of molecular interaction, and can be produced using different techniques. The overall length and composition of the linker can vary. The individual atoms of the linker can include atoms such as carbon, nitrogen, silicone, fluorine, oxygen, sulfur, hydrogen, and phosphorous.

Depending upon the selected linker and nanoparticle, the linker can attach to the nanoparticle by electrostatic interaction or the formation of a covalent bond. Similarly, depending upon the selected linker and fiber, the linker can attach to the nanoparticle by electrostatic interaction or the formation of a covalent bond.

In certain embodiments, a linker is attached to a nanoparticle by a covalent bond. In further embodiments, the linker is attached to both a nanoparticle and fiber by covalent bonds.

The linker can comprise different types of groups or polymers such as polyethylene glycol (PEG), polyaminoacids, polyacrylamides, polyvinylpyrrolidon, zwitterionic polymers, polysaccharides, poly(N-(2-hydroxypropyl)methacrylamide), poly(oligo(ethylene glycol) methyl ether methacrylate), carboxylic dextran, hydrocarbon chains and substituted hydrocarbon chains.

A linker molecule, and associated chemistry resulting in formation of the linker, serves as a bridge between a nanoparticle and fiber allowing for stable and specific binding of the nanoparticle to the linker that preferably enhances the effectiveness of the array.

The linker can also provide a way to control the orientation of the nanoparticles on the substrate, which can be important for certain applications such as electrochemical or catalysts. Additionally, linkers can allow for the tuning of the binding strength between the nanoparticles and substrate, which can be helpful for applications such as using biosensors for analyte detection.

Linkers between the nanoparticle and the fiber can be produced, for example, by functionalizing the nanoparticle and the fiber and then combining the functionalized groups.

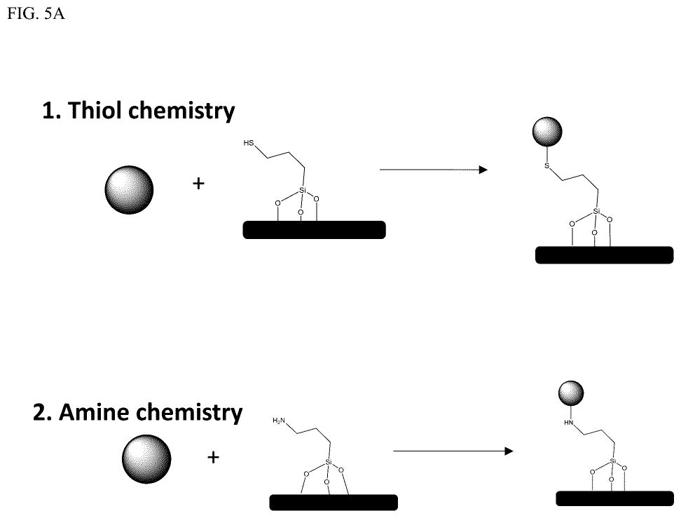

A variety of different chemistries and functional groups can be used to join the nanoparticle to a fiber through a linker. FIG. 3 illustrates some examples of nanoparticle functionalization. The functionalized nanoparticle can combine with an appropriately functionalized fiber. FIG. 4 provides some examples illustrating different linkages produced from different nanoparticle and fiber functionalization.

Linkers can contain additional groups than illustrated in FIG. 3 and FIG. 4, for example, additional groups such as polymers can be present and alternative groups functionalizing the fiber and/or nanoparticle can be employed.

In certain embodiment the linker is joined to the fiber through a “silane coupling group”. A “silane coupling group” comprises one or more attachment site though Si—O₁₋₃, where Si—O₁₋₃ represents multiple S—O bonding, involving the same Si, but different O.

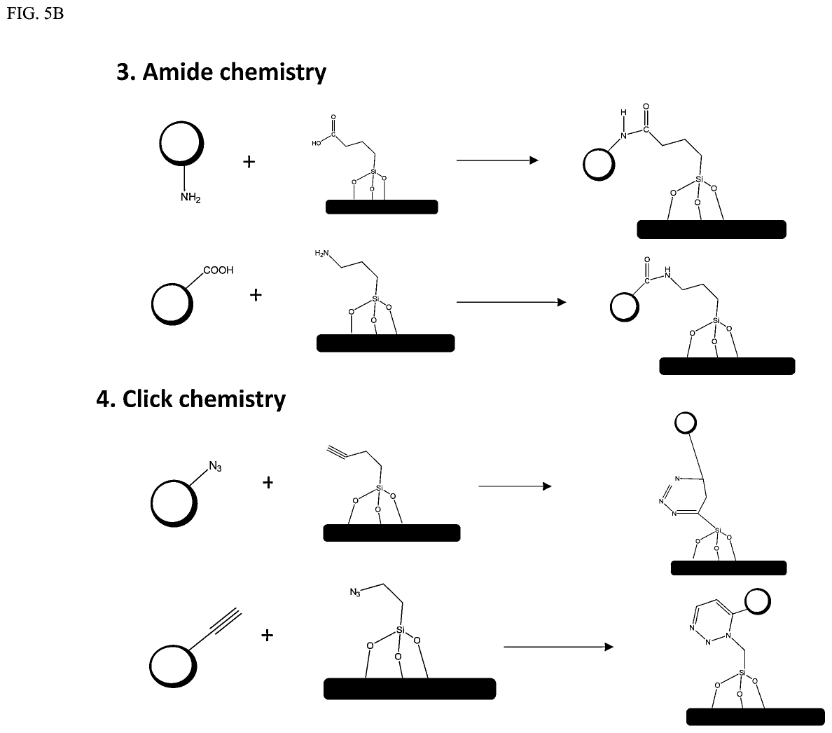

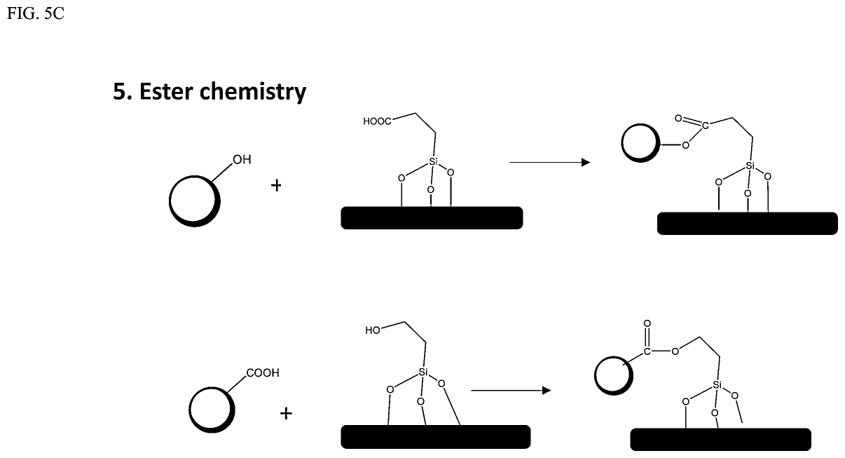

FIGS. 5A, 5B and 5B illustrate attachment of nanoparticle to a fiber using different types of chemistry. FIG. 5A illustrates thiol and amine chemistry, FIG. 5B illustrates amide and click chemistry, and FIG. 5C illustrates ester chemistry. For illustration purposes, the linker is attached to the fiber through Si groups. In the case of a glass fiber, attachment can be through a siloxane bond. Other types of fibers can also be functionalized with silane modifiers to provide for Si—O bonding to the fiber. Silane modifiers are coupling agents providing for the formation of one or more Si—O attachment to the fiber. In different embodiments the silane modifier provides for Si—O or Si—O₂₋₃ (where Si—O₂₋₃ represents multiple S—O bonding, involving the same Si, but different O.

In certain embodiment the silane modifiers comprises X(CH₂)ₙSi—R₃ wherein one or more R₃ is a hydrolysable group and the remaining R₃ groups are independently optionally substituted alk or aryl; X is a functional group reactive with organic material; and n is an integer between 1 and 15. Reference to “alk” indicates is a saturated, monovalent, hydrocarbon chain containing 1-5 carbons. Reference to “aryl” indicates an optionally substituted phenyl. In different embodiments the phenyl is not substituted or substituted. Examples of alk are methyl, ethyl and propyl. Examples of hydrolysable groups include O-alk (e.g., methoxy, ethoxy, propoxy, and butoxy), and O-aryl (e.g., phenoxy).

Ahangaran and Navarchian, Advances in Colloid and Interface Science 286 (2020), 102298, provide examples of silane modifiers for use with metal oxide nanoparticles. In certain embodiments, the silane modifiers provided in Ahangaran and Navarchian are used for fiber functionalization. In a further embodiment, the fiber is a glass fiber.

In certain embodiments, silane modifier group used for fiber functionalization is as provided in Table 2.

```
TABLE 2
  2-((2-(trimethoxysilyl)ethyl)thio)acetic acid
  2-(trimethoxysilyl)propanoic acid
  triethoxysilylpropylmaleamic acid
  hydroxymethyltriethoxysilan
  n-(trimethoxysilylpropyl)ethylenediaminetri-
acetate, tripotassium salt
  2-(trimethoxylsilyl)ethanol
  n-(triethoxysilylpropyl)-o-polyethylene oxide urethane
  hydroxymethoxyl
trimethylsilane
  (hydroxyethoxyethoxy)-t-butyldi-
methylsilane
  hydroxymethyltriethoxysilan
  [hydroxy(polyethyleneoxy)propyl]triethoxysilane,
(8-12 eo)
  2-(trimethoxylsilyl)ethanol
  (hydroxyethoxyethoxy)-t-butyldimethylsilane
  [hydroxy(polyethyleneoxy)propyl]
triethoxysilane, (8-12 eo)
  n-(triethoxysilylpropyl)-o-polyethylene
oxide urethane
  ethynyltripropoxystannane
  ethynyltriethoxyylsilane
  3-aminopropyl)triethoxysilane
  n-(2-aminoethyl)-3-aminoisobutylmethyl-
dimethoxysilane
  n-dimethyl-1,1,1-triphenoxy-
silanamine
(n,n-diethylaminomethyl)
trimethoxysilane,
  n-(2-aminoethyl)-3-aminoisobutyl-
methyldimethoxysilane
  n-(2-aminoethyl)-3-aminopropylmethoxyldiethoxysilane
  ethynyltrimethylsilane
  3-mercaptopropyl-
trimethoxysilane
  3-mercaptopropyltriethoxy-
silane
  3-mercaptopropyltrimethoxysilane
  3-mercaptopropyltriphenoxysilane
  aminoethylaminomethyl)phenethyl-
trimethoxysilane
```

In certain embodiments, silane modifiers are used with fibers containing —OH groups. In a further embodiment, the fiber comprises silicone oxide and forms a siloxane bond with the linker. In certain embodiments, fibers are derivatized to add an —OH group or alternative chemistry is used.

In certain embodiments, the linker is about 20 Da to about 500 kDa.

In certain embodiments, the linker comprises PEG. Different types of PEG can be employed such as linear and branched of varying size. In different embodiments, PEG has a molecular weight of 20 Da to 500,000 Da; a molecular weight of about 200 Da, about 300 Da, or about 400 Da.

In certain embodiments the linker comprises dextran. Dextran of different sizes can be used. In different embodiments, dextran has a size of about 5 kDa to about 75 kDa, or about 45 kDa to about 75 kDa.

In certain embodiments the PEG acts as a linker (anchors to the nanoparticle) and/or spacer. In certain embodiments PEG is a spacer and is attached to the nanoparticle through another moiety. For example, the linker has a —SH moiety attached directly to the nanoparticle and is immobilized onto nanoparticle via Au—SH bond.

Additional linkers and linker chemistry is described in Section II infra.

### II. Analyte Binding Molecule

An analyte binding molecule binds to an analyte and can facilitate separating the analyte from other material present in a sample and/or fixing the analyte in the vicinity of the nanoparticle. Specific or specifically binding refers to the ability to bind to the analyte based upon the structure of the analyte, and distinguish (e.g., bind to a significantly greater extent) the targeted analyte from other analytes naturally present in a sample (e.g., biological sample). Absolute specificity while very helpful for some applications, may not be required. For example, the analyte binding molecule can be used in conjunction with a detection molecule, both of which are specific for the same analyte and assist in detecting the presence of the analyte. In different embodiments the binding molecule has a specificity to a target analyte at least 10×, at 100× or at least 1000× greater than other analytes present in the sample being tested.

A variety of different types of analytes can be detected with appropriate binding molecules. Examples of different types of analytes that can be detected include protein, polypeptide, exosome, nucleic acid, virus, cell, tissue, polysaccharide, lipoprotein, lipid, and therapeutic compounds.

In certain embodiments, the analyte binding molecule is a single-stranded oligonucleotide. A single-stranded oligonucleotide comprises purine or pyrimidine nucleobases or derivatives thereof, able to hydrogen bond via Watson-Crick hydrogen bonds with nucleobases present in DNA or RNA. Naturally occurring DNA and RNA contain a purine (guanine, cytosine, and the less common hypoxanthine) or a pyrimidine (thymine, uracil, or adenine) nucleobase, and a backbone made up of a ribose (RNA) or 2′-deoxyribose (DNA) joined together by phosphodiester groups.

Various modifications can be made to the different polynucleotide components to provide for modified oligonucleotides able to hydrogen bond to DNA or RNA having complementary nucleotide sequences. Examples of purine modifications include 2,6-diaminopurine, 3-deaza-adenine, 7-deasa-guanine, and 8-azido adenine. Examples of pyrimidine modifications include 2-thio-thymidine, 5-carboxamine-uracil, 5-methyl-cytosine, 3-ethynyl-uracil. Examples of phosphodiester modifications include methylphosphonate, phosphorothioate, and guanidinopropyl phosphoramidate. Examples of phosphate replacement includes triazole and guanidinium. Examples of sugar modifications include 2′-modifications such as 2′-F, 2′-methoxy, 2′-amino, and 2′-azido; locked sugar; 3′ end modifications; and 5′ end modification. (Ochoa and Milam, Molecules 2000, 25, 4689, hereby incorporated reference herein in its entirety.)

Another example of a modification includes peptide nucleic acid, where the sugar-phosphate backbone is replaced with a neutral pseudopeptide backbone. The peptide nucleic acid retains nucleobase complementarity, and the ability to hybridize to complementary DNA and RNA. Peptide nucleic acid can be produced, for example, by replacing the phosphodiester backbone with N-(2 aminoethyl) glycine, wherein the nucleobases are connected to the backbone via a methylene carbonyl linker. Additional peptide nucleic acid structures and design consideration are provided in Brodyagin et al., J. Org. Chem. 2021, 17, 1641-1688; and Moccia et al., Artif. DNA:PNA& XNA. 2014 5(3):e1107176; each of which are hereby incorporated by reference herein in their entirety.

Oligonucleotide binding molecules can vary in size and degree of complementarity to target DNA or RNA. The degree of complementarity providing for specificity varies depending on oligonucleotide structure, for example, purine versus pyrimidine nucleobases, and modifications to the nucleobase, sugar and/or phosphodiester group; and the reaction conditions. In different embodiments concerning the binding oligonucleotide molecule size, the oligonucleotide comprises at least 10, at least 12, at least 15, at least 20, at least 30, at least 40, or at least 50, nucleobases. In different embodiments concerning the degree of complementarity to a target nucleic acid analyte, the oligonucleotide binding molecule comprises a region of 10 or more, 11 or more, 12 or more, 13 or more, 15 or more, 16 or more, 17 or more, 18 or more, 19 or more, 20 or more, 21 or more, 22 or more, 23 or more, 24 or more, or 25 or more nucleobases with at least 90%, at least 95% or 100% complementarity to a target nucleic acid analyte.

In certain embodiments the analyte binding molecule is an antibody or comprises a binding fragment of an antibody. An antibody binding fragment contains three complementary determining regions in a variable region framework providing for antigen binding. Examples of binding fragments include FAb fragments, single chain variable region fragments (scFV), single domain fragments (dAbs), Fv fragment, camelid heavy-chain variable domains (VHHs), mini-body and diabody.

In certain embodiments the analyte binding molecule is an aptamer. Aptamers are small single-stranded oligonucleotides with a three-dimensional structure enabling specific binding to a target. The aptamer can be made up of naturally occurring nucleotides or can have one or more modifications. Aptamers can bind to a variety of targets including bacteria, viruses, proteins, toxins, cells (e.g., cancer cells) and tissues. Initial aptamer selection can be carried out using combinatorial oligonucleotide libraries through in vitro selection and iteration processes, such as Systematic Evolution of Ligands by Exponential Enrichment (SELEX). (Kulabhausan et al., Pharmaceutics 2020, 12, 646; and Adachi and Nakamura, Molecules 2019, 24, 4229; both of which are hereby incorporated by reference herein in their entirety.)

In certain embodiments the analyte binding molecule is a protein. Different types of protein can be used to bind to analytes, such an enzyme for binding to a substrate, a substrate for binding to an enzyme, a receptor for binding to a ligand, and a ligand for binding to a receptor.

In certain embodiments the analyte binding molecule is a ligand. Ligands may or may not be a protein.

The analyte binding molecule can be anchored directly to the nanoparticle. Depending on the nanoparticle, anchoring can be achieved through electrostatic interaction or covalent bonds.

Examples of nanoparticle surface immobilization chemistries include: (1) covalent coupling of nanoparticles with linker thiol groups; (2) covalent coupling of active ester-modified nanoparticles with linker amino groups; (3) coupling of maleimide-functionalized nanoparticles with thiol groups; (4) click reaction between dibenzocyclooctyne-modified nanoparticles and azido groups linker; (5) electrostatic adsorption of negatively charged nanoparticles and positively charged linker group; and (6) biotin-NeutrAvidin-mediated linkages.

The length and composition of the analyte binding molecule linker can vary. The individual atoms of the linker can include atoms such as carbon, nitrogen, silicone, fluorine, oxygen, sulfur, hydrogen, and phosphorous.

The linker can comprise different types of groups or polymers such as polyethylene glycol (PEG), polyaminoacids, polyacrylamides, polyvinylpyrrolidon, zwitterionic polymers, polysaccharides, poly(N-(2-hydroxypropyl)methacrylamide), poly(oligo(ethylene glycol) methyl ether methacrylate), carboxylic dextran, hydrocarbon chains and substituted hydrocarbon chains.

In certain embodiments, the linker comprises PEG. Different types of PEG can be employed such as linear and branched of varying sizes. In certain embodiments the PEG is less than about 5K.

In certain embodiments the PEG links directly to the binding molecule where it acts as a linker (anchors to the nanoparticle) and spacer.

In certain embodiments the PEG is a spacer and is attached to the nanoparticle through another moiety. For example, the linker has a —SH moiety attached directly to the nanoparticles and is immobilized onto nanoparticles via Au—S bond.

Additional linkers and linker chemistry is described in Section I.E. supra.

### III. Analyte Capture Molecule

An analyte capture molecule comprises an analyte binding molecule and can be attached to the nanoparticle surface. Analyte binding molecules, attachment to a nanoparticle, different chemistry and different embodiments of analyte binding molecules are described in Section II. supra. Analyte binding molecule can comprise additional groups. In certain embodiments, the analyte capture molecule further comprises one member of a BRET or FRET pair.

In certain embodiments an analyte capture molecule comprises either a donor or an acceptor energy transfer molecule. Multiply analyte capture molecules containing different donor or acceptor energy transfer molecules may be present. For example, different donor molecules may be present on different capture molecules (containing different analyte binding) providing for multiplexing; or different acceptor molecules may be present on different capture molecules (containing different analyte binding) providing for multiplexing.

### IV. Analyte Detection Molecule

An analyte detection molecule comprises a second analyte binding molecule and a molecule providing a detectable signal, where the first analyte binding molecule is that present in the analyte capture molecule. The first and second analyte binding molecules bind to a particular analyte at different locations, such that binding of the first analyte binding molecule to an analyte does not prevent binding of the second analyte binding molecule to the analyte. The detection molecule should be able to move in solution allowing the second analyte binding molecule to contact and bind to analyte.

Analyte binding molecules, that can serve as the first and second binding molecules are described in Section II supra. Section II supra also provides for attachment to a nanoparticle, different chemistry and different embodiments of analyte binding molecules.

Energy transfer pairs comprise provide two different molecules, where one molecule can transfer energy to second molecule, and detection of the energy from the second molecule can be detected. In certain embodiments, the detection molecule (1) comprises one member of an energy transfer pair; or (2) comprises both members of an energy transfer pair. In cases where the detection molecule comprises one member of an energy transfer pair, the second member can be provided, for example, by the nanoparticle or a group attached to the nanoparticle, by a secondary detection molecule that binds to the analyte detection molecule, or by the analyte capture molecule. In further embodiments the energy transfer pair provides for a fluorescent, chemiluminescent, or bioluminescence signal.

In certain embodiments, the detection molecule further comprises an enzyme able to cleave a substrate to produce a colorimetric change. In further embodiments, the enzyme is horse radish peroxidase or alkaline phosphatase; and/or the second binding molecule is an antibody or antigen binding fragment thereof.

In certain embodiments, the analyte detection molecule is a signal amplifier. A “signal amplifier” comprises (a) an analyte binding molecule; and (b) a localized surface plasmon resonance nanostructure and either (i) a bioluminescence resonance energy transfer (BRET) assembly complex, or (ii) fluorescence resonance energy transfer (FRET) assembly complex.

Different configurations of an analyte binding molecule, localized surface plasmon resonance nanostructure, and the BRET assembly complex or (FRET) assembly complex are possible, such as: (1) the analyte binding molecule and the BRET or FRET assembly complexes are both linked to the localized surface plasmon resonance nanostructure; (2) the localized surface plasmon resonance nanostructure and BRET or FRET assembly are both linked to the antigen binding molecule; and (3) the localized surface plasmon resonance nanostructure and the analyte binding molecule are both linked to the BRET or FRET assembly complex.

A localized surface plasmon resonance nanostructure can enhance the BRET and FRET signals. The closer the localized surface plasmon resonance nanostructure to the BRET and FRET assembly complex the greater the enhancement. Preferably, the localized surface plasmon resonance nanostructure is within about 5 to about 20 nm of a BRET or FRET member producing a signal being detected. In certain embodiments the localized surface plasmon resonance nanostructure is 12, 13, 14, 15, 16 nm from the BRET or FRET assembly complex.

A localized surface plasmon resonance nanostructure can also enhance other types of signals such as a colorimetric signal produced by horseradish peroxidase. The plasmonic structure should be about 5 to about 20 nm from the emission. Du et al., (2014) ACS Nano 2014 8 (10), 9964-9969.

In certain embodiments a detection capture molecule comprises either a donor or an acceptor energy transfer molecule. Multiply analyte detection molecules containing different donor or acceptor energy transfer molecules may be present. For example, different donor molecules may be present on different detection molecules (containing different analyte binding) providing for multiplexing; or different acceptor molecules may be present on different analyte capture molecules (containing different analyte binding) providing for multiplexing.

### V. BRET Pair

A BRET pair is made up of a luciferase donor and an acceptor fluorophore. The pair can be joined to the same or different molecule. In certain embodiments, the BRET pair is provided in a BRET assembly complex, where the complex comprises a luciferase donor joined to an acceptor fluorophore through a luciferase donor-acceptor linker.

Following oxidation of a luciferase substrate, energy is transferred from the luciferase donor to excite the fluorophore acceptor. Emission from the luciferase donor and fluorophore can be measured. Emission can be characterized by, for example, wavelength, intensity, lifetime, and polarization.

Energy transfer between the luciferase donor and fluorophore acceptor occurs when the donor and acceptor are within approximately <10 nm of each other. The closer the donor and acceptor are to each other the stronger the emission.

In certain embodiments, the distance between luciferase donor and fluorophore acceptor is about 3 nm, about 4, nm, about 5 nm, about 6 nm, about 7 nm, about 8 nm, about 9 nm, or about 10 nm.

The luciferase donor-acceptor linker can be made up of atoms such as carbon, nitrogen, silicone, oxygen, sulfur, hydrogen, fluorine, and phosphorous,

The linker can comprise different types of groups or polymers such as PEG, polyaminoacids, polyacrylamides, polyvinylpyrrolidon, zwitterionic polymers, polysaccharides, poly(N-(2-hydroxypropyl)methacrylamide), poly(oligo(ethylene glycol) methyl ether methacrylate), carboxylic dextran, hydrocarbon chains and substituted hydrocarbon chains.

The linker provides a stable structure and can be made up of different groups joined together by different types of molecular interactions, and can be produced using different techniques. For example, the BRET assembly can contain a luciferase molecule joined to a HaloTag protein, where the HaloTag protein is linked to a fluorophore molecule. The overall distance between the luciferase molecule and fluorophore, taking into the diameter of the haloTag protein joined to the luciferase donor (about 3.3 nm, see, e.g., Yazaki et al., Nucleic Acids Research, Volume 48, Issue 2, 24 Jan. 2020, Page e8) and the 12 atom linker (about 1.2 nm) to the fluorophore is about 4.5 nm. Alternative techniques for joining different groups include EDC/NHS coupling, click chemistry, streptavidin-biotin and complementary oligonucleotides.

Multiple variations of BRET have been developed using different luciferase donors, substates, and fluorophore acceptors. (See, e.g., Dale et al., Front. Bioeng. Biotechnol. 2019, 7, 56, hereby incorporated by reference herein in its entirety.) Examples of different variations are shown in Table 3.

TABLE 3

| Luciferase Donor | | Donor Emission | | Acceptor emission |
|---|---|---|---|---|
| (Enzyme) | Substrate | (nm) | Fluorophore | (nm) |
| RLuc | Coelenterazine | 480 | eYFP | 530 |
| RLuc | Coelenterazine 400a | 395 | GFP | 510 |
| | (Deep Blue C ™) | | | |
| RLuc8 | Coelenterazine 400a | 395 | GFP | 510 |
| | (Deep Blue C ™) | | | |
| Firefly | Luciferin | 565 | DsRed | 583 |
| RLuc/RLuc8 | Coelenterazine | 480 | QDot | 605 |
| NanoLuc ®-HT | Furimazine | 460 | HaloTag ® | 618 |
| | | | Ligand | |
| NanoLuc ®-HT | Furimazine | 460 | HaloTag ® | 516 |
| | | | Ligand | |

GFP refers to green florescent protein, eYFP refers to enhanced yellow florescent protein, and RLuc refers to Renilla luciferase. NanoLuc®-HT is a nanoluciferase, also referred to as Nluc. Furimazine is 2-furanylmethyl-deoxy-coelenterazine.

Nluc was derived from the 19 kDa subunit of a larger multi-component luciferase isolated from the deep sea shrimp Oplophorus gracilirostris. The luminescence of the 19 kDa subunit was enhanced through mutagenesis and numerous coelenterazine analogs were screened to optimize the substrate. (Dale et al., Front. Bioeng. Biotechnol. 2019, 7, 56.)

Examples of additional luciferase donor/substrates include teLuc and diphenylterazine. TeLuc and diphenylterazine are derivatives of Nluc and coelenterazine. (Dale et al., Front. Bioeng. Biotechnol. 2019, 7, 56.)

Examples of additional fluorophores and a fluorophore system includes boron-dipyrromethene (BODIPY), alprenolol-tetramethylrhodamine (TAMRA), 4-nitro-7-aminobenzofurazan, Alexa Fluor™ and the HaloTag® fluorophore system. The HaloTag® system is made up of a small halotag protein (33 kDa) fused to a chosen protein and a chloroalkane linker, wherein the linker is joined to a fluorophore. (Dale et al., Front. Bioeng. Biotechnol. 2019, 7, 56.)

In certain embodiments, the BRET assembly comprising the luciferase donor conjugated to the fluorophore acceptor is selected from the following combinations: NLuc-HT/HL-Oregon green, RLuc/YFP, RLuc/florescent protein (GFP), RLuc8/GFP, firefly luciferase/DsRed, RLuc/ODot, Rluc8/ODot, and NanoLuc/Halotag-florescent ligand.

### VI. FRET Pair

A FRET pair is made up of a fluorophore donor and an acceptor fluorophore. The pair can be joined to the same or different molecule. In the certain embodiments, the FRET pair is provided as a FRET assembly complex, where the FRET assembly complex comprise a donor fluorophore joined to an acceptor fluorophore through a fluorophore donor-acceptor linker.

Following excitation of the donor fluorophore, energy is transferred from the donor to excite the fluorophore acceptor. Emission from the donor and acceptor fluorophores can be measured. Emission can be characterized by, for example, wavelength, intensity, lifetime, and polarization.

Energy transfer between the donor fluorophore and acceptor fluorophore occurs when the donor and acceptor are within approximately <10 nm of each other. (Bajar et al., Sensors (Basel). 2016 Sep. 14; 16(9): 1488.) The closer the donor and acceptor are to each other the stronger the emission. In different embodiments, the distance is about 1 nm, about 2 nm, about 3 nm, about 4 nm, about 5 nm, about 6 nm, about 7 nm, about 8 nm, or about 10 nm.

The fluorophore donor-acceptor linker can include atoms such as carbon, nitrogen, silicone, oxygen, sulfur, fluorine, hydrogen, and phosphorous.

The linker can comprise different types of groups or polymers such as PEG, polyaminoacids, polyacrylamides, polyvinylpyrrolidon, zwitterionic polymers, polysaccharides, poly(N-(2-hydroxypropyl)methacrylamide), poly(oligo(ethylene glycol) methyl ether methacrylate), carboxylic dextran, hydrocarbon chains and substituted hydrocarbon chains.

Multiple combinations of FRET donor and acceptor fluorophores have been developed. Examples of different FRET pairs include: cyan florescent protein and yellow florescent protein; green florescent protein and red florescent protein; and far-red florescent protein and infrared florescent protein. Examples of cyan florescent protein and yellow florescent protein include Aquamarine, ECFP, mTurquoise2, mCerulean3, mTFP1, EYFP, m Venus, SEYFP, mCitrine, and YPet. Examples of green florescent protein and red florescent protein include EGFP, NowGFP, Clover, mClover3, mNeonGreen, mRuby2, mRuby3 and mCherry. Examples of far-red florescent protein and infrared florescent protein include mPlum, eqFP650 and mCardinal. (Bajar et al. florescent protein hypertext://doi.org/10.3390/s16091488, hereby incorporated by reference herein in its entirety).

### VII. Analyte Detection Assays

Different assays can be performed taking in to account the composition of a particular array and the use of additional components. Examples of different types of methods include use of localized surface plasmon resonance (LSPR), surface enhanced Raman spectroscopic applications (SERS), enzyme-linked immunoassay applications (ELISA), Fourier transform infrared spectroscopic (FTIR), bioluminescence, fluorescence, and chemiluminescence and paper-based mass spectrometry.

In certain embodiments, the fiber array does not comprise an analyte capture molecule and the nanoparticle is a plasmonic nanostructure. In such embodiments the fiber array can provide for general signal amplification due to the plasmonic nanostructure provided on the array. For example, the fiber array can be provided to a well functionalized for an ELISA assay.

In certain embodiments, the fiber array comprises an analyte capture molecule and is used in methods further comprising the use of a detection molecule. In further embodiments, detection is based on resonance, energy transfer, such as provided by surface plasmon resonance, bioluminescence, fluorescence, or chemiluminescence. In a further embodiment, an ELISA format is used where the detection molecule is an antibody further comprising an enzyme able to cleave a substrate to produce a colorimetric change. In further embodiments, the enzyme is horse radish peroxidase or alkaline phosphatase.

In certain embodiments, the detection us carried out an energy transfer pair. Different formats can be employed. In certain embodiments, (1) the detection molecule comprises one member of an energy transfer pair; or (2) comprises both members of an energy transfer pair. In cases where the detection molecule comprise one member of an energy transfer pair, the second member can be provided, for example, by the nanoparticle or a group attached to the nanoparticle, by a secondary detection molecule that binds to the analyte detection molecule, or by an analyte capture molecule. In further embodiments the energy transfer pair provided for a fluorescent, chemiluminescent, or bioluminescence signal.

### VII.A Energy Transfer Pairs Provided on Different Molecules

In certain embodiments the assay is perform using a first analyte binding molecule comprising a first energy transfer molecule and a second analyte binding molecule comprising a second energy transfer molecule. The two energy transfer molecules provide for an energy transfer pair. The site where the analyte binding molecules bind, and the position of the energy transfer molecule on the binding molecules, should be selected to facilitate energy transfer.

In different embodiments, the analyte binding molecules are each attached to an independently selected nanoparticle. In further embodiments, each nanoparticle is an independently selected plasmonic nanoparticle such as provided in section I.A supra and/or an independently semiconductor such as provided in section I.B supra.

In further embodiments, the first and second energy transfer pair is a FRET or BRET pair such as that provided in sections V or VI supra. Preferably, the distance between FRET and BRET energy transfer pairs, when each binding molecules is bound to antigen, is within approximately <10 nm of each other. The closer the donor and acceptor are to each other the stronger the emission. In certain embodiments, the distance between energy transfer pairs are about 3 nm, about 4, nm, about 5 nm, about 6 nm, about 7 nm, about 8 nm, about 9 nm, or about 10 nm.

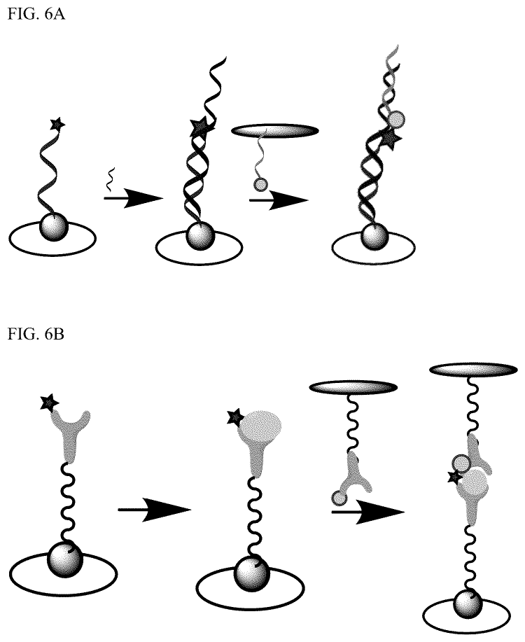

FIG. 6A and FIG. 6B illustrates detection using energy transfer molecules present on different analyte binding molecules. FIG. 6A illustrates a nucleic acid capture molecule attached to the fiber though a spherical plasmonic nanoparticle, wherein the nucleic acid comprises a first energy transfer molecule (star); and a nucleic acid detection molecule attached to a plasmonic nanorod, wherein the nucleic acid comprises a second energy transfer molecule (circle). FIG. 6B illustrates an antibody capture molecule attached to the fiber though a spherical plasmonic nanoparticle, wherein the antibody comprises a first energy transfer molecule (star); and an antibody detection molecule attached a plasmonic nanorod, wherein the antibody comprises a second energy transfer molecule (circle) containing the second energy transfer molecule. In both FIG. 6A and FIG. 6B, analyte binding positions the energy transfer molecule to facilitate energy transfer.

### VII.B BRET or FRET Assembly Complex

In certain embodiment, detection is carried out using a BRET or FRET assembly complex. In further embodiments, the assay involves the use of a BRET assembly complex and comprises: (a) analyte binding to an analyte capture molecule, where the capture molecule is attached to nanoparticle and the nanoparticle is attached to the nanofiber array; (b) providing an signal amplifier comprising a second analyte binding molecule binding to the analyte; (c) adding a luciferase substrate; and (d) measuring fluorescence, localized surface plasmon resonance, bioluminescence, or surface-enhanced Raman scattering. More than one readout can be measured. Unless otherwise indicated, reference to steps (a) and (b) do not provide for a particular order. In certain embodiments, step (a) is followed by step (b). In certain embodiments, step (b) is followed by step (a).

In further embodiments, the assay involves the use of a FRET assembly complex and comprises: (a) analyte binding to an analyte capture molecule, where the capture molecule is attached to nanoparticle and the nanoparticle is attached to the fiber array; (b) providing a signal amplifier comprising a second analyte binding molecule binding to the analyte; (c) exciting the fluorophore; and (d) measuring fluorescence or surface-enhanced Raman scattering. More than one readout can be measured. Unless otherwise indicated, reference to steps (a) and (b) do not provide for a particular order. In certain embodiments, step (a) is followed by step (b). In certain embodiments, step (b) is followed by step (a).

In different embodiments the analyte is quantified based on two or more different readouts selected from fluorescence, bioluminescence, localized surface plasmon resonance and surface-enhanced Raman scattering; or measuring at least fluorescence and/or bioluminescence. Dual techniques can increase overall accuracy, to confirm a positive result, and are particularly useful where low amounts of analyte are present.

In certain embodiments, unbound amplifier is removed prior to the measuring step.

In certain embodiments, analyte from a particular sample are either purified or not purified prior to the assay; and/or analyte from a particular sample are either amplified or not amplified prior to the assay.

### VIII. Nanoparticle Combinations

Certain aspects and embodiments are directed to a nanoparticle combination comprising:
(a) a first plasmonic nanoparticle comprising a first analyte binding molecule that specially binds to analyte, wherein the first analyte binding molecule further comprises a first energy transfer molecule, and
(b) a second plasmonic nanoparticle comprising a second analyte binding molecule that specially binds to the analyte, wherein the second analyte binding molecule further comprises a second energy transfer molecule,
wherein either the first energy transfer molecule is a donor and the second energy transfer molecule is an acceptor or the first energy transfer molecule is an acceptor and second energy transfer molecule is a donor.

The nanoparticle combination can be provided together, for example, in a kit; or be provided separately. In certain embodiments, the first plasmonic nanoparticle is attached to a fiber array through a linker. In certain embodiments, the first plasmonic nanoparticle is attached without a linker.

In certain embodiments, the nanoparticle combination is used for detecting the presence or amount of an analyte in a sample. In different embodiments, the first plasmonic nanoparticle is a plasmonic analyte capture molecule comprising a first energy transfer molecule and is attached to a fiber array (with or with a linker); and the second plasmonic nanoparticle is a plasmonic analyte detection molecule comprising a second energy transfer molecule free in solution.

The first and second transfer molecules provide for an energy transfer pair. The site where the analyte binding molecule binds and the position of the energy transfer molecule on the capture analyte binding molecule should be selected to facilitate energy transfer. In different embodiments each nanoparticle is an independently selected plasmonic nanoparticle as provided in section I.A supra.

In further embodiments, the first and second energy transfer pair is a FRET or BRET pair such as that provided in sections V or VI supra. Preferably, the distance between FRET and BRET energy transfer pairs, when each binding molecules is bound to antigen, is within approximately <10 nm of each other. The closer the donor and acceptor are to each other the stronger the emission. In certain embodiments, the distance between energy transfer pairs are about 3 nm, about 4, nm, about 5 nm, about 6 nm, about 7 nm, about 8 nm, about 9 nm, or about 10 nm.

A kit refers to a physical structure housing one or more components. A kit typically includes packaging material, a label or packaging insert including a description of the components or instructions.

Labels or inserts can include identifying information of one or more components and instructions for use. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

### IX. Additional Aspects and Embodiments

Additional aspects, embodiments, and combinations thereof include the following:
1) A fiber array comprising a plurality of nanoparticles attached to one or more fibers, wherein each nanoparticle of the plurality of the nanoparticles is attached to a fiber of the one or more fibers at a different location through a linker wherein at least one of either:
   a) the linker is attached to the fiber through a silane coupling group;
   b) the nanoparticle further comprises an analyte capture molecule;
   c) the linker is covalently attached to the nanoparticle;
   d) the linker is 20 Da to 500 KDa; and/or
   e) the fiber array comprises at least two fibers.
2) The fiber array of 1, comprising 5 or more fibers.
3) The fiber array of 1 or 2, wherein the fiber array is a three-dimensional fiber array.
4) The fiber array of 3, wherein the fiber array has a height of about 0.2 mm to about 1 mm.
5) The fiber array of any one of 1-4, wherein the nanoparticle is a plasmonic nanoparticle.
6) The fiber array of 5, wherein the plasmonic nanoparticle comprise a metal selected from rhenium, ruthenium, rhodium, palladium, silver, osmium, iridium, platinum, gold, and alumni; and/or a non-metal selected from graphene, silica, and carbon nanotube.
7) The fiber array of 6, wherein the plasmonic nanoparticle comprise gold.
8) The fiber array of any one of 1-4, wherein the nanoparticle is polymeric nanoparticle, or polymeric vesicle; or comprises either a liposome, micelle, peptide, or dendrimer.
9) The fiber array of any one of 1-4, wherein the nanoparticle comprises a quantum dot.
10) The fiber array of any one of 1-9, wherein the fiber comprises cross-linked silicone oxide or a polymer. In further embodiments, the fiber comprises cross-linked silicone oxide and the linker is attached to the fiber through a siloxane bond.
11) The fiber array of 10, wherein the one or more fibers are optical transparent.
12) The fiber array of 11, wherein the one or more fibers provide a three-dimensional glass fiber array.
13) The fiber array of any one of 1-10, wherein a fiber of the fiber array comprises a polymer, wherein the polymer comprises polyethersulfone, polydimethylsiloxane, nylon, polypropylene, polylactic acid, cellulose, polycarbonate, polyacrylamide, polyacylonitrile, polyvinyl alcohol, cellulose acetate, polyvinyl chloride, polyamine/polyurethane, polyvinylidene fluoride, polyethylene terephthalate, polymethyl methacrylate, polystyrene, epoxy resin, thermoplastic polyurethane, poly(lactic-co-glycolic acid), polyether amine, or polyvinylpyrrolidone.
14) The fiber array of any one of 1-13, wherein the average fiber diameter of the one or more fibers is 0.1 μm to 50 μm.
15) The fiber array of any one of 1-14, wherein the plurality of the nanoparticles provides for about 4% to about 95% coverage over a length of at least 14 μm² of the one or more fibers.
16) The fiber array of 15, wherein the plurality of the nanoparticles provides for about 50% to about 75% coverage over a length of at least 25 μm² of the one or more fibers.
17) The fiber array of any one of 1-16, wherein the plurality of the nanoparticles comprises 1000 nanoparticles.
18) The fiber array of any one of 1-17, wherein the one or more fibers is produced by electrospinning.
19) The fiber array of any one of 1-18, wherein the linker is attached to the fiber through a silane coupling group.
20) The fiber array of any one of 1-19, wherein the linker comprises one or more group or polymer selected from polyethylene glycol (PEG), polyaminoacids, polyacrylamides, polyvinylpyrrolidon, zwitterionic polymers, polysaccharides, poly(N-(2-hydroxypropyl)methacrylamide), poly(oligo(ethylene glycol) methyl ether methacrylate, carboxylic dextran, hydrocarbon chain and/or substituted hydrocarbon chain.
21) The fiber array of claim 20, wherein the linker comprises a polyethylene glycol having a molecular weight of about 20 Da to about 500 kDa.
22) The fiber array of any one of claims 1-21, wherein the linker comprises a group from Table 2.
23) The fiber array of any one of 1-18 or 20-22, wherein the linker is covalently bonded to the fiber through a sulfur, amine, amide, carboxylate, or phosphene.
24) The fiber array of claim 23, wherein the linker is covalently bonded to the fiber through a sulfur.
25) The fiber array of any one of 1-24, wherein the one or more fibers comprises at least two different fibers having a different refractive index.
26) The fiber array of any one of 1-25, wherein each nanoparticle of the plurality of the nanoparticles is in a shape of a disk, rod, star, triangle and/or spherical.
27) The fiber array of any one of 1-26, wherein the nanoparticle further comprises an analyte capture molecule.
28) The fiber array of 27, wherein the analyte capture molecule comprises either a single-stranded oligonucleotide, antibody or binding fragment thereof, or an aptamer.
29) The fiber array claim 27 or 28, wherein the analyte capture molecule further comprises an energy transfer molecule. In a further embodiment, the energy transfer molecule is a fluorophore or luciferase molecule
30) A method of detecting the presence of an analyte in a sample comprising the step of contacting the fiber array of any one of 27-29, with the sample, wherein the analyte capture molecule specifically binds to the analyte, and measuring bound analyte. In further embodiment, spectrometric emission is measured.
31) The method of 30, wherein the analyte capture molecule is attached to a first plasmonic nanoparticle and further comprises a first energy transfer molecule; and measuring bound analyte comprises using an analyte detection molecule, wherein the analyte detection molecule specifically binds to the analyte, comprises a second energy transfer molecule and is attached to a second plasmonic nanoparticle. The first plasmonic nanoparticle and the second nanoparticle can be the same or different. In certain embodiments, the first plasmonic nanoparticle and the second nanoparticle are different, for example in size, shape, and/or composition. In certain embodiments the first and second energy transfer molecule are a BRET pair or a FRET pair.
32) The method of 30, wherein the capture molecule specifically binds to the antigen and the method further comprises using an analyte detection molecule that specifically binds to the analyte, wherein the analyte detection molecule further comprises a fluorophore, an enzyme, BRET pair or a FRET pair.
33) The method of 32, wherein the enzyme is luciferase forming a BRET pair with a fluorophore present on the analyte capture molecule.
34) The method of 32, wherein the enzyme is either horse radish peroxidase or alkaline phosphatase.
35) The method of any of 30-34, wherein detecting spectrometric emission comprises measuring localized surface plasmon resonance, fluorescence, bioluminescence, chemiluminescence, surface enhanced Raman, Fourier transform infrared spectroscopy, surface enhanced bioluminescence, or paper-mass spectroscopy.
36) A nanoparticle combination comprising
   a) a first plasmonic nanoparticle comprising a first analyte binding molecule that specially binds to analyte, wherein the first analyte binding molecule further comprises a first energy transfer molecule, and
   b) a second plasmonic nanoparticle comprising a second analyte binding molecule that specially binds to the analyte, wherein the second analyte binding molecule further comprises a second energy transfer molecule, wherein either the first energy transfer molecule is a donor and the second energy transfer molecule is an acceptor or the first energy transfer molecule is an acceptor and the second energy transfer molecule is a donor.
37) The nanoparticle combination of 36, wherein the first and second energy transfer molecule is FRET or BRET pair.
38) The nanoparticle combination of 36 or 37, wherein the first plasmonic nanoparticle is attached to a fiber array through a linker.
39) The nanoparticle combination of 36 or 37, wherein the first plasmonic nanoparticle is attached to a fiber array without a linker.

Examples are provided below further illustrating different features of the present invention and methodology for practicing the invention. The provided examples do not limit the claimed invention.

### EXAMPLES

### Example 1: Three-Dimensional Array

Example 1 illustrates techniques that can used to produce a three-dimensional array using gold nanoparticles and fiberglass. Other types of nanoparticles of different sizes and shapes, other types of fibers and alterative chemistry, as illustrated, for example, throughout the application. Nanoparticles were produced based on the procedures provided by Masterson et al., (2020) Anal. Chem. 92, 13, 9295-9304, hereby incorporated by reference here.

AuSNP-Citrate: AuSNPs (spherical) were synthesized using a published procedure (Masterson et al., (2020) Anal. Chem. 92, 13, 9295-9304) with slight modification to provide a larger volume. Briefly, a 10 ml HAuCl₄H₂O solution (4 mg/mL) was added to 390 mL H₂O in a 2 L round bottom flask on a heating mantle. The temperature was then slowly increased until the solution started to boil, then 3 mL of aqueous solution of sodium citrate (10 mg/mL) was quickly injected. The reaction was allowed to proceed for 5 minutes while stirring, and then the heat source was removed, and the red solution was allowed to cool.

AuNR-CTAB: The initial step of the AuNR (nanorod) seed solution production added 250 μL of 10 mM HAuCl₄ solution to 7.5 mL of 100 mM cetyltrimethylammonium bromide (“CTAB”) solution in a 20 mL glass vial at room temperature and stirring at medium speed. After five minutes, 600 L of 10 mM NaBH₄ solution was injected and the reaction was continue for an additional 15 minute. NaBH₄ acts as a reducing agent, and after adding NaBH₄ a yellow to light color change appears (seed solution).

AuNR solution was prepared by adding 2 mL of 10 mM HAuCl₄ solution to 28 ml of 100 mM CTAB solution, mixing well, adding 0.4 ml of 10 mM AgNO₃, and adding 0.22 ml of 100 mM L. Ascorbic acid and mixing well. The reduction of HAuCl₄ was confirmed via the formation of a colorless solution.

Finally, 48 μL of the seed solution was injected in the AuNR solution and the reaction was complete for at least four hours.

AuTNP-TEA/CTAB: AuTNP (trianges) were chemically synthesized using a previously developed procedure with minor modification. Briefly, Et₃PAu(I)Cl (18.0 mg, 0.05 mmol) was dissolved in 40 mL of CH₃CN and stirred for 10 minutes at room temperature. Next, 0.038 mL of triethylamine (“TEA”) was injected into the solution, and the temperature was gently raised. At 40° C., 600 μL of poly(methylhydrosiloxane) average MW of 1700-3200 Daltons (“PMHS”) was added, the reaction was allowed to proceed with slow magnetic stirring, and the temperature was raised to 50° C. During the reaction, the color of the solution changed from colorless to pink, purple, and light blue. The reaction stopped at a 690 nm UV peak.

Array Preparation: Three dimensional micro membranes were obtained using glass fiber sheets with or without binders and nitrocellulose sheets. Sheet parameters were basis weight 75 g/m², callaper 0.43 mm, wicking rate 5 (s/2 cm), and water absorption 79 mg/cm². The substrate was cut to the desired shape using Carbon Steel Hollow Punch Set Kit and or custom-made dies to accommodate the well plates diameters as shown in Table 4.

TABLE 4

| | Microwell | | |
|---|---|---|---|
| Multi-Well | Diameter | | |
| Plates | (mm) | Cutter size (mm) | |
| 96 well plates | 5 | 4.7625 | mm |
| 48 well plates | 6 | 5.7625 | mm |
| 24 well plates | 10, 13 | 9.7625, 12.7625 | mm |
| 12 well plates | 10, 14 | 9.7625, 13.7625 | mm |
| 6 well plates | 10, 14, 20 | 9.7625, 13.7625, 19.7625 | mm |

After a particular size was obtained, cutouts were washed with isopropyl alcohol and methanol, sonicated for 1 hour at a moderate speed and dried in the vacuum oven at 80° C. to remove organic solvents. This step helps remove the excess microfibers and clean the glass fiber's surface before functionalization.

The substrate was then reacted with 40%3-aminopropyl-triethoxysilane (“APTES”) in isopropyl alcohol (“IPA”)/ethanol for 5 hours in a sealed container on a rotary shaker. Excess APTES was removed, and the substrate was cleaned in IPA/ethanol five times with 10-minute shaking intervals. After the excess APTES was removed, the fiber arrays were dried under a vacuum oven at 120° C.

Nanoparticles functionalized with Polyethylene Glycol Thiol (PEG(1k)-SH): AuSP-citrate, AuTNP-TEA/CTAB, AuNRS-CTAB, and AuNST-CTAB were separately subjected to ligand exchange with PEG-SH. Accordingly, 10 mM-PEG-SC (N-hydroxysuccinimide)-SH per 1 optical density (OD) nanoparticle solution (NPS) was dissolved in Tri-HCl buffer at pH 3.0, and the reaction was continued for 3 hours. PEG functionalized nanoparticles were separated via solvent extraction using CHCl₃.

Self-Assembly: PEGlyated nanoparticles, 20 mL of 3 OD solution, was added to 100 fiber arrays in a 50 mL centrifuge tube and placed on a rotary shaker at a medium speed for 12 hours. Chips were separated and then washed with copious amounts of chloroform. During the final wash, fiber arrays were sonicated at a medium speed in a cold bath for one hour to remove loosely bound nanoparticles. The 2D nanoparticles on the 3D fiber glass array were then ready to use.

### Example 2: Analyte Detection

The fiber array was further modified with analyte capture molecules comprising analyte binding molecules targeting bladder cancer antigens. Analyte detection molecules comprising analyte binding molecules targeting bladder cancer antigens and a BRET assembly complex were used to illustrate detection of different analytes and generate a calibration curve with known analyte amounts.

Materials and Reagents: HAuCl₄, sodium citrate (99.5%), hexadecyltrimethylammonium bromide (CTAB, 99%), D-(+)-Glucose (99.5%), sodium borohydride, silver nitrate (99%), L(+)-ascorbic acid (reagent grade), 3-aminopropyl-triethoxysilane (APTES, 94%) were purchased from Sigma-Aldrich. Thiolated polyethylene glycols were purchased from BIOCHEMPEG. Single stranded oligonucleotides modified 3′-SH—(CH₂)₃-ssDNA, microRNAs, and RNase H enzymes were purchased from Integrated DNA Technologies. All chemicals were used without further purifications. RNase-free sterile water and PBS buffer (pH 7.2) were obtained from Sigma-Aldrich. Corning 96-multiwell plates were purchased from Sigma-Aldrich.

Nucleic acids used as ssDNA analyte binding molecules for the analyte capture molecule and detection molecules are provided in the Table 5. CAP1 indicates functionalized to AuNRs and use as an analyte detection molecule. CAP2 indicates functionalized to Au spheres and use as an analyte capature molecule.

TABLE 5

| | ssDNA (oligomer) sequences used for this study | |
|---|---|---|
| Name | Sequence | Modification |
| 143-CAP1- | 5′-AGC ACT GCA CC-3′ (SEQ ID NO: 1) | /3ThioMC3-D/ |
| SSDNA | | |
| 143-CAP2- | 5′-ACC AGA GAT GC-3′ (SEQ ID NO: 2) | /5ThioMC6-D/ |
| SSDNA | | |
| 205-CAP1- | 5′-GGA ATG AAG GA-3′ (SEQ ID NO: 3) | /3ThioMC3-D/ |
| SSDNA | | |
| 205-CAP2- | 5′-CAG ACT CCG GT-3′ (SEQ ID NO: 4) | /5ThioMC6-D/ |
| SSDNA | | |
| 200c CAP1 | 5′-TGG GTA AGA CG-3′ (SEQ ID NO: 5) | /3ThioMC3-D/ |
| 200C CAP 2 | 5′-CCA AAC ACT GC-3′ (SEQ ID NO: 6) | /5ThioMC6-D/ |
| 16-1 CAP1 5′3 | 5′-ACG TGC TGC TA-3′ (SEQ ID NO: 7) | /3ThioMC3-D/ |
| 16-1 CAP2 5′3′ | 5′-CGC CAA TAT TT-3′ (SEQ ID NO: 8) | /5ThioMC6-D/ |
| UCA CAP1 | 5′-CAC GGA ATG AGG GCC AGG ACA GGG GGC CGG-3′ | /3ThioMC3-D/ |
| | (SEQ ID NO: 9) | |
| UCA CAP2 | 5′ TGA GGA CCC GAG GTC GCA GGT GGA TCT CTT-3′ | /5ThioMC6-D/ |
| | (SEQ ID NO: 10) | |

Micro RNA sequences and a longer non-coding RNA (UCA) sequence used as analytes in generating calibration curves, are provided in Table 6.

TABLE 6

| | microRNA sequences used for this study |
|---|---|
| Name | Sequence |
| miR-143 | 5′-GGU GCA GUG CUG CAU CUC UGG U-3′ (SEQ ID NO: 11) |
| miR-205 | 5′-UCC UUC AUU CCA CCG GAG UCU G-3′ (SEQ ID NO: 12) |
| miR200c | 5′-CGU CUU ACC CAG CAG UGU UUG G-3′ (SEQ ID NO: 13) |
| MiR 16-1 | 5′-UAG CAG CAC GUA AAU AUU GGC G-3′ (SEQ ID NO: 14) |
| UCA full length | 5′-CCG GCC CCC TGT CCT GGC CCT CAT TCC GTG AAG AGA TCC |
| | ACC TGC GAC CTC GGG TCC TCA-3′ (SEQ ID NO: 15) |

Thiol-modified-ssDNAs, microRNAs, mRNAs, and proteins were kept at −20° C., and patient samples were stored at −80° C. NanoLuc®-HaloTag® Protein and Nano-Glo buffers were purchased from Promega Corporation. Washing and blocking buffers were prepared in the lab.

Analytes, the analyte binding molecule present in the capture molecule and detection molecule are noted in Table 7. Material was obtained from commercial sources such as ProSci Inc., Novus Biological and abbexa and Sinobiologics,

TABLE 7

| Analyte | Detection Molecule | Capture Molecule |
|---|---|---|
| miR-205 | Polynucleotide | Polynucleotide |
| miR-16-1 | Polynucleotide | Polynucleotide |
| miR-143 | Polynucleotide | Polynucleotide |
| UCA1 nucleic acid | Polynucleotide | Polynucleotide |
| IGF2 encoding mRNA | Antibody | Antibody |
| ANXA10 encoding mRNA | Antibody | Antibody |
| NMP-22 protein | Antibody | Antibody |
| HCFHrp protein | Antibody | Antibody |
| miR-200C | Polynucleotide | Polynucleotide |
| UPKB encoding mRNA | Antibody | Antibody |
| ABL1 encoding mRNA | Antibody | Antibody |
| CRH encoding mRNA | Antibody | Antibody |

Detection Molecule Functionalization. AuSPs incorporated into the fiber array were functionalized using polyethylene glycol thiol (1K) with n-hydroxysuccinimide (NHS) to provide SC-PEG-S—Au. The LSPR peak of the PEG (1K)-SH functionalized AuSP array was observed at 550 nM. Functionalization was carried out using 1 nM AuSP nanoparticles mixed with a 1:2000 mole ratio of PEG-1K at room temperature. The reaction was allowed to complete for 24 hours on a rotary shaker at medium speed. Excess PEG was removed by ultracentrifugation at 10,000 rpm for 30 minutes. The supernatant was removed and continued for three rounds of cleaning to remove all excess PEG-SH.

ssDNA loading: Ten micromolar ssDNA disulfide was incubated in 100 μM tris(2-carboxyethyl)phosphine) (TCEP) in 1 mL 10×PBS buffer at pH 5.2, at room temperature for one hour while giving 60 second sonications every 15 minutes. Sample was then diluted to the required volume to obtain 10 μM ssDNA disulfide. The fiber array was positioned at the bottom of individual wells of a 96 white well-plate, and loaded with 200 μL 10 μM ssDNA disulfide. The plate was sealed and left in the dark, at room temperature for 12 hours. After the 12 hours, 4 μL of a 3M NaCl solution was added five times at one-hour intervals. During each addition, the solution was sonicated for 10 seconds. Following the final sonification, wells were sealed and incubated for another 12 hours. Blocking buffer (StartingBlock™ (PBS) Blocking Buffer from Thermo fisher) was then added to the fiber array to avoid nonspecific binding, the functionalized array was incubated for 30 minutes, and then fiber arrays were centered and dried at 25° C. for 2 hours before use in the analytical application.

Anti-body Loading: Fiber array containing SC-PEG(1K)-SH functionalized AuSP were subjected to EDC/NHS coupling in 2-(N-morpholino)ethanesulfonic acid (MES) buffer at pH 6.0 to provide COOH activation and then 20 μg/mL antibody solution in conjugate buffer solution was added. The reaction continued for eight hours, and then the fiber array was cleaned using a washing buffer to remove the unbound antibodies from the surface. After removing the unbound antibodies, the fiber array surface was blocked using a blocking buffer.

Gold Nanorods Preparation: AuNRs synthesis was carried out in two different steps. Seed solution was made by adding 250 μL of a 10 mM HAuCl₄ solution to 7.5 mL of a 100 mM CTAB solution in a 20 mL glass vial at room temperature and stirring at medium speed. After five minutes, 600 μL of 10 mM NaBH₄ solution was injected and allowed to react for 15 minutes. NaBH₄ acts as a reducing agent, and after adding NaBH₄, a yellow to light color change appears.

To prepare 250 mL of AuNR solution, 250 mL of a 0.055 M CTAB solution and 17.86 mL of a 10 mM HAuCl₄ solution were combined and mixed well. A solution of 3.57 mL of 10 mM AgNO₃ was added, followed by adding 1.96 mL of 10 mM L-ascorbic acid. The solution was mixed well. Reduction of HAuCl₄ was confirmed via the formation of a colorless solution.

Initially, 342 μL of the seed solution was injected into the AuNR solution, and the reaction was allowed to complete for at least four hours. The color changed from a colorless light wine to a brownish black, indicating the formation of AuNRs. After AuNRs formation, excess CTAB was removed by centrifuging two times. The obtained AuNRs had a wavelength of about 680 nm with an average size of ˜ 40 nm.

AuNR PEG Functionalization: AuNRs were redissolved in Tris buffer at pH 3.0, combined with a 1 mM/1 OD SC-PEG(1K)-SH:NH₂-PEG(1K)-SH 1:1 solution, and allowed to react for 3 hours. PEG functionalized AuNRs were extracted to chloroform using the solvent-solvent extraction method, after which the chloroform was fully evaporated.

Nucleic Acid Loading on to AuNR: Reactions were carried out in 20 mL scintillation glass vials which were cleaned using 12 M NaOH. A solution of 10 μM of ssDNA CAP2 was mixed with 100 μM TCEP solution in PBS buffer 1 mL at pH 5.2 for one hour. After 1 hour, the mixture was diluted in a PBS 10× buffer at pH 8.0 containing a 1 OD solution of SC-PEG (1K)-SH:NH₂-PEG (1K)-SH functionalized AuNRs. The reaction was continued for 12 hours, then 20 μL of 3M NaCl was added five times dropwise (total addition was 1 ml) at one hour intervals. After each addition, the solution was sonicated 10 seconds. After completion of NaCl addition, the reaction was left in a dark place for 24 hours before use.

Antibody Loading on to AuNR: SH-PEG (1K)-SC and NH₂-PEG (1K)-SH functionalized AuNRs were coupled with maleimide MA-PEG (1K)-NH₂ in PBS buffer 10× at pH 8.0 for 4 hours. Separated AuNRs were reactivated using EDC/NHS coupling, and a 20 μg/mL antibody solution was added. The reaction continued for eight hours at room temperature in a conjugate buffer, and the AuNRs were purified via centrifugation.

ssDNA Loading on to AuNR: PEG-Lyated (SC-PEG (1K)-SH:NH₂-PEG (1K)-SH) AuNRs were subjected to ligand exchange with single-stranded oligonucleotides (ssDNA). Single-stranded oligonucleotides were obtained in disulfide form, and before the ligand exchange, disulfide bonds were reduced using TCEP. Reduction was carried out on 10 μM ssDNA disulfide using 100 μM TCEP in acetate buffer, 500 mM, pH 5.2 at room temperature for 1 hour and a 60 second sonication every 15 minutes. After the disulfide was reduced to thiol, the solution was added to a mixture containing a 1 OD/1 mL AuNRs solution in 10 mM phosphate buffered saline (PBS) at pH 8.0 in 12 M NaOH treated glass vials. After gentle shaking by hand, the reaction was allowed to complete for another 12 hours.

After complete reaction, a 20 μl of 3 M NaCl solution was added dropwise to each vial with gentle handshaking, followed with 10 second sonication. NaCl helps reduce the repulsion forces between the two strands, increasing the ssDNA loading onto the nanoparticles. Vials were stored in a drawer (dark room temperature) for at least 24 hours before use.

BRET Assembly: Nine microliters of a 9 μg/μl solution of Nluc-HT enzyme was reacted with 0.6 μL of 1 mM Oregon green dye in the presence of 150 μL of 10% IGEPAL® CA-630, in 2 mL of 1×PBS buffer in a dark 20 mL scintillated vial. The reaction continued in a cold room on a rotating shaker at a medium speed for 16 hours. The Nluc-HT-HL-Oregon tagged molecule was directly used with no further purification.

Nluc-HT-Oregon BRET molecule preparation: After ssDNA/mRNA/protein antibodies were conjugated to AuNRs, the AuNRs were coupled with MA-PEG-NH₂ in PBS buffer 10× at pH 8.0 for 4 hours. AuNRs were separated via centrifugation, blocked on the surface using blocking buffer, and purified again via centrifugation. A 100 μL Nluc-HT-HL-Oregon BRET preassembled 1 OD/mL solution was injected to AuNRs in 4-(2-hydroxyethyl)-1-piperazineethanesulfonic acid (HEPES) buffer at pH 8.0, and the reaction was continued for 3 hours at 4° C. After 3 hours AuNRs coupled Nluc-HT-HL-Oregon assembly was purified using 100 MW cutoff filters.

Detection Assay: The detection assay was evaluated using different amounts of analytes added to urine. Assays were carried out using 96 well plates. Each well was prepared to detect a specific biomarker. Calibration was carried out using analytes ranging in amounts from 100 ng/ml to 100 ag/mL.

AuSP assembled wells were functionalized with either ssDNA CAP1 to capture nucleic acid or antibody to capture proteins, followed by blocking the free substrate to avoid nonspecific binding. Then 100 μL of a human urine sample, centrifuged at 3000 rpm for 15 minutes to remove all large cell debris, was mixed with 100 μL of hybridization/coupling buffer, then directly added to each well and incubated for 4 hours. Analyte capture molecules captured the specific biomarkers directly from the urine without any purification or amplification. The use of polyethylene glycol thiol as a spacer and a blocking agent eliminated unspecific molecule binding. The 96 well plate was washed with washing buffer and dried under N₂ Gas, 200 μL of blocking buffer was again added to avoid further nonspecific attachment, and let sit for 30 minutes at room temperature.

A 100 μL solution of 1 OD signal amplifier was added to the target well, which had already captured the target analyte. Signal amplifiers comprised a BRET assembly carrying AuNRs solution, having ssDNA CAP2 for nucleic acid assay or antibody for the protein assay. The reaction was allowed to continue in either hybridization or coupling buffer for 4 hours. Excess AuNRs were removed, and the wells washed with washing buffer to remove all unbound AuNRs from the solution.

Finally, the well plate was placed in a GLOMAX Discover plate reader, and 100 μL of reaction buffer containing furimazine (3 μL: 10 mL) was injected into each well followed by 20 second shaking of the well plate, and the generated luminescence, and fluorescence was reported.

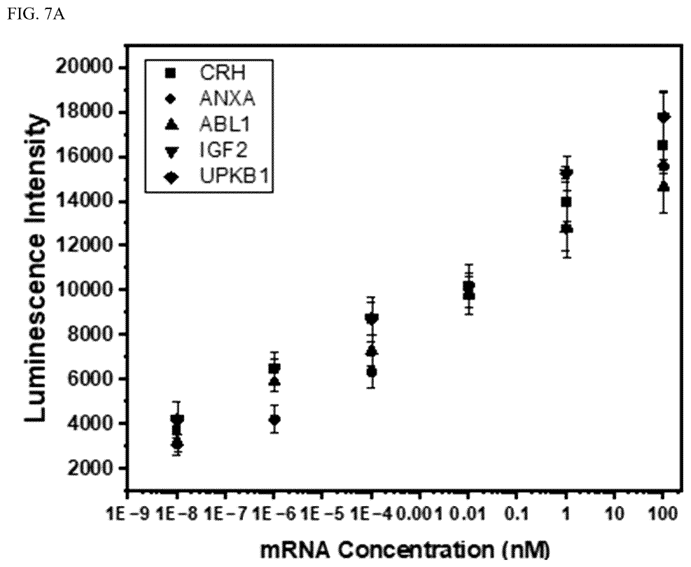

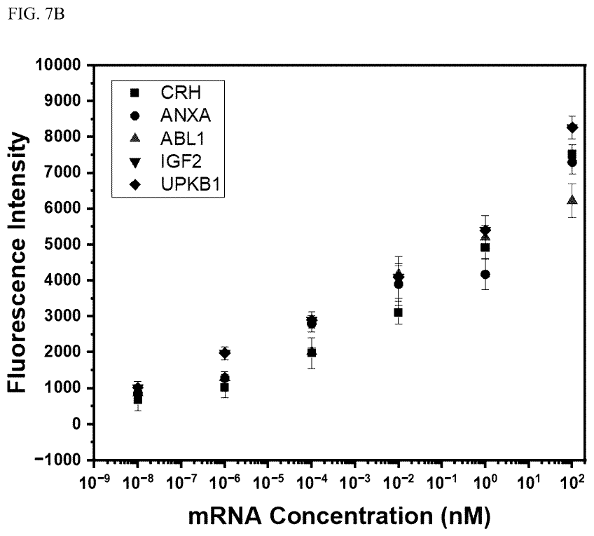

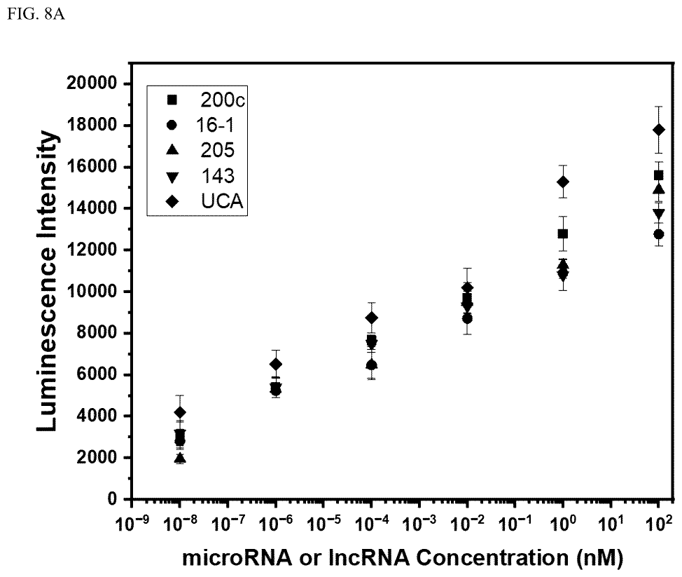

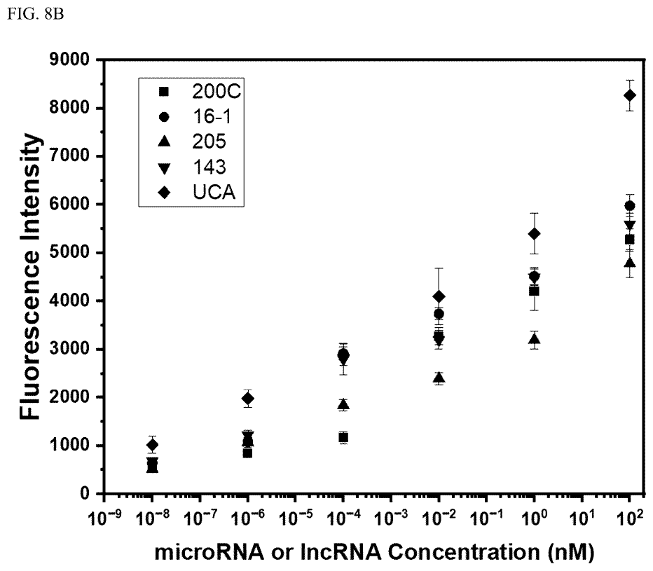

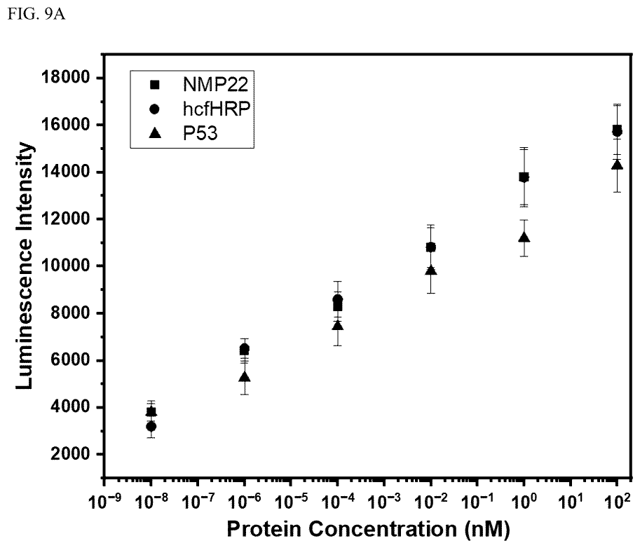

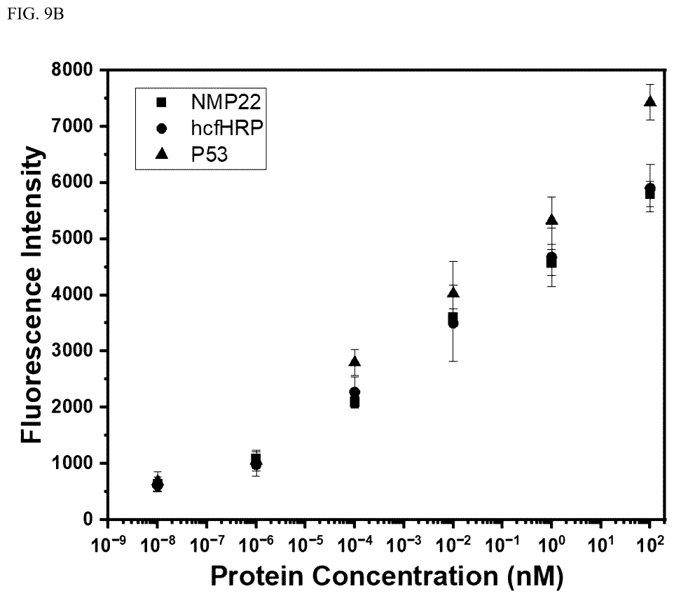

Results: Detection of analytes in the tested range of 100 ng/ml to 100 ag/ml was mostly linear. FIGS. 7A, 7B, 8A, 8B, 9A and 9B show calibration plots measuring luminescence and fluorescence for different analytes. FIG. 7A illustrates CRH, ANAX10, ABL1, IGF2 and UPKB1 luminescence. FIG. 7B illustrates CRH, ANAX10, ABL1, IGF2 and UPKB1 fluorescence. FIG. 8A illustrates miR-200c (200c), miR-16-1 (16-1), miR-205 (205), miR-143 (143) and long non-coding RNA (UCA) luminescence. FIG. 8B illustrates miR-200c (200c), miR-16-1 (16-1), miR-205 (205), miR-143 (143) and long non-coding RNA (UCA) fluorescence. FIG. 9A illustrates NMP22, HCFHrp (hcfHRP) and P53 luminescence. FIG. 9B NMP22, HCFHrp (hcfHRP) and P53 fluorescence.

Tables 8-13 provide individual data points along with the standard deviation.

TABLE 8

| Conc (nm) | CRH | std | ANXA | std | IGF2 | std | ABL1 | Std | UPKB1 | std |
|---|---|---|---|---|---|---|---|---|---|---|
| 100 | 16500 | 1240 | 15600 | 1140 | 17800 | 1184 | 14670 | 1200 | 17800 | 1120 |
| 1 | 13980 | 895 | 12800 | 1275 | 15300 | 275 | 12800 | 980 | 15300 | 775 |
| 0.01 | 10200 | 945 | 9850 | 945 | 10200 | 945 | 9800 | 850 | 10200 | 945 |
| 1.00E−04 | 8700 | 1020 | 6350 | 725 | 8750 | 720 | 7300 | 680 | 8750 | 720 |
| 1.00E−06 | 6430 | 488 | 4220 | 625 | 6520 | 688 | 5900 | 420 | 6520 | 688 |
| 1.00E−08 | 3700 | 720 | 3100 | 472 | 4200 | 820 | 3200 | 475 | 4200 | 820 |

Row intensity values obtained for mRNA luminescence assay.

TABLE 9

| Conc (nm) | CRH | std | ANXA | std | IGF2 | std | ABL1 | Std | UPKB1 | std |
|---|---|---|---|---|---|---|---|---|---|---|
| 100 | 7520 | 275 | 7300 | 320 | 8270 | 320 | 6230 | 470 | 8270 | 320 |
| 1 | 4920 | 295 | 4175 | 420 | 5400 | 420 | 5200 | 340 | 5400 | 420 |
| 0.01 | 3100 | 320 | 3900 | 580 | 4100 | 580 | 4180 | 230 | 4100 | 580 |
| 1.00E−04 | 1980 | 422 | 2800 | 225 | 2900 | 225 | 2015 | 120 | 2900 | 225 |
| 1.00E−06 | 1025 | 280 | 1285 | 180 | 1980 | 180 | 1300 | 102 | 1980 | 180 |
| 1.00E−08 | 680 | 295 | 875 | 175 | 1020 | 175 | 870 | 78 | 1020 | 175 |

Row intensity values obtained for mRNA fluorescence assay

TABLE 10

| Conc (nm) | 200C | std | 16-1 | std | 205 | std | 143 | std | UCA | std |
|---|---|---|---|---|---|---|---|---|---|---|
| 100 | 15600 | 675 | 12780 | 555 | 14920 | 565 | 13800 | 480 | 17800 | 1120 |
| 1 | 12800 | 820 | 10900 | 248 | 11300 | 290 | 10800 | 750 | 15300 | 775 |
| 0.01 | 9700 | 740 | 8720 | 752 | 9520 | 520 | 9300 | 425 | 10200 | 945 |
| 1E−4 | 7650 | 275 | 6480 | 624 | 6490 | 725 | 7500 | 275 | 8750 | 720 |
| 1E−6 | 5420 | 498 | 5240 | 325 | 5340 | 240 | 5400 | 478 | 6520 | 688 |
| 1E−8 | 3100 | 620 | 2810 | 397 | 1970 | 225 | 3200 | 590 | 4200 | 820 |

Row intensity values obtained for mRNA luminescence assay.

TABLE 11

| Conc (nm) | 200C | std | 16-1 | std | 205 | std | 143 | std | UCA | std |
|---|---|---|---|---|---|---|---|---|---|---|
| 100 | 5270 | 228 | 5980 | 225 | 4780 | 287 | 5600 | 225 | 8270 | 320 |
| 1 | 4200 | 384 | 4520 | 175 | 3200 | 184 | 4500 | 175 | 5400 | 420 |
| 0.01 | 3270 | 178 | 3740 | 125 | 2400 | 125 | 3200 | 188 | 4100 | 580 |
| 1E−4 | 1170 | 125 | 2920 | 132 | 1840 | 120 | 2800 | 320 | 2900 | 225 |
| 1E−6 | 840 | 85 | 1080 | 75 | 1070 | 105 | 1220 | 105 | 1980 | 180 |
| 1E−8 | 620 | 122 | 640 | 97 | 520 | 77 | 680 | 78 | 1020 | 175 |

Row intensity values obtained for microRNA and lncRNA fluorescence assay.

TABLE 12

| Conc (nm) | NMP22 | std | hcfHrp | std | P53 | std |
|---|---|---|---|---|---|---|
| 100 | 15800 | 1040 | 15725 | 1175 | 14285 | 1120 |
| 1 | 13800 | 1175 | 13785 | 1257 | 11200 | 775 |
| 0.01 | 10800 | 845 | 10820 | 945 | 9800 | 945 |
| 1E−4 | 8300 | 625 | 8600 | 758 | 7450 | 820 |
| 1E−6 | 6420 | 525 | 6520 | 428 | 5275 | 720 |
| 1E−8 | 3800 | 372 | 3200 | 472 | 3800 | 480 |

Row intensity values obtained for Protein luminescence assay.

TABLE 13

| Conc (nm) | NMP22 | std | hcfHrp | std | P53 | std |
|---|---|---|---|---|---|---|
| 100 | 5800 | 220 | 5900 | 420 | 7430 | 320 |
| 1 | 4575 | 230 | 4675 | 520 | 5325 | 420 |
| 0.01 | 3600 | 154 | 3500 | 680 | 4025 | 580 |
| 1E−4 | 2100 | 105 | 2275 | 275 | 2800 | 225 |
| 1E−6 | 1085 | 132 | 980 | 200 | 1055 | 180 |
| 1E−8 | 640 | 120 | 612 | 108 | 687 | 175 |

Row intensity values obtained for Protein fluorescence assay.

Table 14 summarizes equations for calibration plots for sample containing different amounts of analytes in urine. The LODs were calculated by measuring the relevant intensity for the blank sample and then calculating the Z (mean+3σ) value. The Z value was then converted into the relative concentration using the calibration curve. Blank samples were made up of nanoparticles functionalized with spacer molecule (SC-PEG(1K)-SH), but with capture molecule present. Signal amplifier solution was added and after four hours solution was removed and cleaned with washing buffer, and luminescence and fluorescence intensity were obtained by adding the substrate. The first set (left side) of Table 14 provides bioluminescence, and the second set (right side) provides fluorescence.

TABLE 14

| | | | LOD | | | | LOD |
|---|---|---|---|---|---|---|---|
| Analyte | LR Equation | R² | (aM) | Analyte | LR Equation | R² | (aM) |
| CRH1 | y = 546.9ln(x) + | 0.9892 | 0.93 | CRH1 | y = 297.71ln(x) + | 0.9974 | 105.23 |
| | 13696 | | | | 5024 | | |
| ANXA | y = 587.48ln(x) + | 0.9892 | 0.47 | ANXA | y = 258.51ln(x) + | 0.9974 | 48.23 |
| | 13203 | | | | 4942.5 | | |
| ABL1 | y = 499.75ln(x) + | 0.9929 | 0.52 | ABL1 | y = 252.29ln(x) + | 0.9632 | 102.5 |
| | 12397 | | | | 5042 | | |
| IGF2 | y = 543.02ln(x) + | 0.9972 | 0.05 | IGF2 | y = 280.55ln(x) + | 0.9911 | 481 |
| | 14172 | | | | 5118 | | |
| UPKB1 | y = 545.631n(x) + | 0.9929 | 0.01 | UPKB1 | y = 273.61ln(x) + | 0.9916 | 1126 |
| | 14127 | | | | 4942 | | |
| 200c | y = 507.75ln(x) + | 0.9984 | 2.72 | CRH1 | y = 258.51ln(x) + | 0.9974 | 69.70 |
| | 13277 | | | | 4942.5 | | |
| 16-1 | y = 447.14ln(x) + | 0.9967 | 1.13 | 16-1 | y = 264.49ln(x) + | 0.9979 | 50.59 |
| | 11264 | | | | 4822 | | |
| 205 | y = 465.32ln(x) + | 0.9954 | 0.73 | 205 | y = 247.98ln(x) + | 0.9962 | 132.78 |
| | 12528 | | | | 4606 | | |
| 143 | y = 472.14ln(x) + | 0.9979 | 0.46 | 143 | y = 235.82ln(x) + | 0.996 | 70.62 |
| | 12698 | | | | 4470 | | |
| UCA | y = 490.97ln(x) + | 0.9899 | 1.12 | UCA | y = 225.94ln(x) + | 0.9996 | 161.99 |
| | 10719 | | | | 3136.5 | | |
| NMP | y = 525.12ln(x) + | 0.9968 | 5.94 | NMP | y = 258.51ln(x) + | 0.9964 | 27.89 |
| | 13447 | | | | 4622.5 | | |
| HCFHrp | y = 537.531n(x) + | 0.9949 | 6.38 | BTA | y = 265.79ln(x) + | 0.9997 | 740.46 |
| | 13488 | | | | 4690 | | |
| P53 | y = 528.44ln(x) + | 0.9931 | 0.01 | P53 | y = 292.61ln(x) + | 0.996 | 323.21 |
| | 13790 | | | | 5234.5 | | |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

I/We claim:

1. A fiber array comprising a plurality of nanoparticles attached to one or more fibers, wherein each nanoparticle of the plurality of the nanoparticles is attached to a fiber of the one or more fibers at a different location through a linker wherein at least one of either:
a) the linker is attached to the fiber through a silane coupling group;
b) the nanoparticle further comprises an analyte capture molecule;
c) the linker is covalently attached to the nanoparticle;
d) the linker is 20 Da to 500 KDa; and/or
e) the fiber array comprises at least two fibers.

2. The fiber array of claim 1, comprising 5 or more fibers.

3. The fiber array of claim 2, wherein the fiber array is a three-dimensional fiber array.

4. The fiber array of claim 3, wherein the nanoparticle is a plasmonic nanoparticle.

5. The fiber array of claim 4, wherein the plasmonic nanoparticle comprise a metal selected from rhenium, ruthenium, rhodium, palladium, silver, osmium, iridium, platinum, gold, and alumni; and/or a non-metal selected from graphene, silica, and carbon nanotube.

6. The fiber array of claim 4, wherein the plasmonic nanoparticle comprise gold.

7. The fiber array of claim 2, wherein the nanoparticle is polymeric nanoparticle, or polymeric vesicle; comprises either a liposome, micelle, peptide, or dendrimer; or comprises a quantum dot.

8. The fiber array of claim 4, wherein the fiber comprises cross-linked silicone oxide or a polymer.

9. The fiber array of claim 2, wherein the one or more fibers provides a three-dimensional glass fiber array.

10. The fiber array of claim 4, wherein the fiber comprises a polymer, wherein the polymer comprises polyethersulfone, polydimethylsiloxane, nylon, polypropylene, polylactic acid, cellulose, polycarbonate, polyacrylamide, polyacylonitrile, polyvinyl alcohol, cellulose acetate, polyvinyl chloride, polyamine/polyurethane, polyvinylidene fluoride, polyethylene terephthalate, polymethyl methacrylate, polystyrene, epoxy resin, thermoplastic polyurethane, poly(lactic-co-glycolic acid), polyether amine, or polyvinylpyrrolidone.

11. The fiber array of claim 8, wherein the average fiber diameter of the one or more fibers is 0.1 μm to 50 μm.

12. The fiber array of claim 8, wherein the plurality of the nanoparticles provides for about 4% to about 95% coverage over a length of at least 14 μm² of the one or more fibers.

13. The fiber array on claim 4, wherein the plurality of the nanoparticles provides for about 50% to about 75% coverage over a length of at least 25 μm² of the one or more fibers.

14. The fiber array of claim 4, wherein the plurality of the nanoparticles comprises 1000 nanoparticles.

15. The fiber array of claim 4, wherein the linker is attached to the fiber through a silane coupling group.

16. The fiber array of claim 15, wherein the linker comprises a polyethylene glycol having a molecular weight of about 20 Da to about 500 kDa.

17. The fiber array of claim 16, wherein the linker is covalently bonded to the fiber through a sulfur.

18. The array of claim 4, wherein the nanoparticle further comprises an analyte capture molecule.

19. The fiber array of claim 18, wherein said analyte capture molecule further comprises an energy transfer molecule attached to the analyte capture molecule.

20. The fiber array of claim 19, wherein the analyte capture molecule comprises either a single-stranded oligonucleotide, antibody or binding fragment thereof, or an aptamer; and the energy transfer molecule is a fluorophore or luciferase molecule.

21. The fiber array of claim 1, wherein
a) the nanoparticle is attached to the fiber through a siloxane bond;
b) the fiber array comprises 5 or more fibers;
c) the fiber array is a three-dimensional glass fiber array;
d) the nanoparticle is a plasmonic nanoparticle;
e) the linker comprises a polyethylene glycol having a molecular weight of about 20 Da to about 500 kDa;
f) the plurality of the nanoparticles provides for about 4% to about 95% coverage over a length of at least 14 μm² of the one or more fibers;
g) the average fiber diameter of the one or more fibers is 0.1 μm to 50 μm; and
h) an analyte capture molecule.

22. The fiber array of claim 21, wherein the analyte capture molecule comprises either a single-stranded oligonucleotide, antibody or binding fragment thereof, or an aptamer; and the analyte capture molecule further comprises a fluorophore or luciferase molecule.

23. A method of detecting the presence of an analyte in a sample comprising the step of contacting the fiber array of claim 19 with the sample, wherein the analyte capture molecule specifically binds to the analyte, and measuring bound analyte.

24. The method of claim 23, wherein the analyte capture molecule is attached to a first plasmonic nanoparticle and further comprises a first energy transfer molecule; and measuring bound analyte comprises using an analyte detection molecule, wherein the analyte detection molecule specifically binds to the analyte, comprises a second energy transfer molecule and is attached to a second plasmonic nanoparticle; and measuring spectrometric emission.

25. The method of claim 24, wherein the first and second energy transfer molecule are a BRET pair or a FRET pair.

26. The method of claim 23, wherein detecting spectrometric emission comprises measuring localized surface plasmon resonance, fluorescence, bioluminescence, chemiluminescence, surface enhanced Raman, Fourier transform infrared spectroscopy, surface enhanced bioluminescence, or paper-mass spectroscopy.

27. A nanoparticle combination comprises:
a) a first plasmonic nanoparticle comprising a first analyte binding molecule that specially binds to analyte, wherein the first analyte binding molecule further comprises a first energy transfer molecule, and
b) a second plasmonic nanoparticle comprising a second analyte binding molecule that specially binds to the analyte, wherein the second analyte binding molecule further comprises a second energy transfer molecule wherein either the first energy transfer molecule is a donor and the second energy transfer molecule is an acceptor or the first energy transfer molecule is an acceptor and the second energy transfer molecule is a donor.
